(19) United States
(12) Patent Application Publication
(10) Pub. No.: US 2010/0297073 A1
(43) Pub. Date: Nov. 25, 2010

(54) HETEROCYCLIC ANTIVIRAL COMPOUNDS

(76) Inventors: Elbert Chin, San Jose, CA (US); Jim Li, San Francisco, CA (US); Albert Sui-Ting Lui, Sunnyvale, CA (US); Francisco Xavier Talamas, Mountain View, CA (US)

Correspondence Address: Grant D. Green, Patent Law Department,;M/S A2-250, Roche Palo Alto LLC, 3431 Hillview Avenue, Palo Alto, CA, 94304, US

(21) Appl. No.: 12/784,432

(22) Filed: May 20, 2010

Related U.S. Application Data
(60) Provisional application No. 61/179,857, filed on May 20, 2009.

(51) Int. Cl. A61K 31/4725; C07D 405/10; C07D 401/10; A61K 31/517; A61K 31/501; A61K 38/21; A61K 38/20; A61K 38/19; A61P 31/14

(52) U.S. Cl. 424/85.2; 546/283.1; 546/141; 544/284; 544/238; 514/337; 514/309; 514/266.21; 514/252.01; 424/85.4; 424/85.1

(57) ABSTRACT

Compounds having the formula I wherein

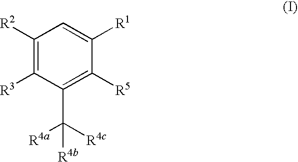

wherein R¹, R², R^{3b}, R^{4a}, R^{4b}, R^{4c} and as defined herein are Hepatitis C virus NS5b polymerase inhibitors. Also disclosed are compositions and methods for treating an HCV infection and inhibiting HCV replication.

### CROSS-REFERENCE TO PRIOR APPLICATIONS

This application claims the benefit of priority to U.S. Ser. No. 61/179,857 filed May 20, 2009 which is hereby incorporated by reference in their entirety.

### FIELD OF THE INVENTION

The present invention provides non-nucleoside compounds of formula I, and certain derivatives thereof, which are inhibitors of RNA-dependent RNA viral polymerase. These compounds are useful for the treatment of RNA-dependent RNA viral infection. They are particularly useful as inhibitors of hepatitis C virus (HCV) NS5B polymerase, as inhibitors of HCV replication, and for the treatment of hepatitis C infection.

### BACKGROUND

Hepatitis C virus is the leading cause of chronic liver disease throughout the world. (Boyer, N. et al., J. Hepatol. 2000 32:98-112). Patients infected with HCV are at risk of developing cirrhosis of the liver and subsequent hepatocellular carcinoma and hence HCV is the major indication for liver transplantation.

HCV has been classified as a member of the virus family Flaviviridae that includes the genera flaviviruses, pestiviruses, and hapaceiviruses which includes hepatitis C viruses (Rice, C. M., Flaviviridae: The viruses and their replication. In: Fields Virology, Editors: B. N. Fields, D. M. Knipe and P. M. Howley, Lippincott-Raven Publishers, Philadelphia, Pa., Chapter 30, 931-959, 1996). HCV is an enveloped virus containing a positive-sense single-stranded RNA genome of approximately 9.4 kb. The viral genome consists of a highly conserved 5′ untranslated region (UTR), a long open reading frame encoding a polyprotein precursor of approximately 3011 amino acids, and a short 3′ UTR.

Genetic analysis of HCV has identified six main genotypes which diverge by over 30% of the DNA sequence. More than 30 subtypes have been distinguished. In the US approximately 70% of infected individuals have Type 1a and 1b infection. Type 1b is the most prevalent subtype in Asia. (X. Forms and J. Bukh, Clinics in Liver Disease 1999 3:693-716; J. Bukh et al., Semin. Liv. Dis. 1995 15:41-63). Unfortunately Type 1 infectious is more resistant to therapy than either type 2 or 3 genotypes (N. N. Zein, Clin. Microbiol. Rev., 2000 13:223-235).

Viral structural proteins include a nucleocapsid core protein (C) and two envelope glycoproteins, E1 and E2. HCV also encodes two proteases, a zinc-dependent metalloproteinase encoded by the NS2-NS3 region and a serine protease encoded in the NS3 region. These proteases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown. It is believed that most of the non-structural proteins encoded by the HCV RNA genome are involved in RNA replication

Currently a limited number of approved therapies are available for the treatment of HCV infection. New and existing therapeutic approaches for treating HCV infection and inhibiting of HCV NS5B polymerase activity have been reviewed: R. G. Gish, Sem. Liver. Dis., 1999 19:5; Di Besceglie, A. M. and Bacon, B. R., Scientific American, October: 1999 80-85; G. Lake-Bakaar, Current and Future Therapy for Chronic Hepatitis C Virus Liver Disease, Curr. Drug Targ. Infect Dis. 2003 3(3):247-253; P. Hoffmann et al., Recent patent on experimental therapy for hepatitis C virus infection (1999-2002), Exp. Opin. Ther. Patents 2003 13(11):1707-1723; M. P. Walker et al., Promising Candidates for the treatment of chronic hepatitis C, Exp. Opin. Investing. Drugs 2003 12(8):1269-1280; S.-L. Tan et al., Hepatitis C Therapeutics: Current Status and Emerging Strategies, Nature Rev. Drug Discov. 2002 1:867-881; J. Z. Wu and Z. Hong, Targeting NS5B RNA-Dependent RNA Polymerase for Anti-HCV Chemotherapy, Curr. Drug Targ.—Infect. Dis. 2003 3(3):207-219.

Ribavirin (1-((2R,3R,4S,5R)-3,4-Dihydroxy-5-hydroxymethyl-tetrahydro-furan-2-yl)-1H-[1,2,4]triazole-3-carboxylic acid amide; Virazole®) is a synthetic, non-interferon-inducing, broad-spectrum antiviral nucleoside analog. Ribavirin has in vitro activity against several DNA and RNA viruses including Flaviviridae (Gary L. Davis. Gastroenterology 2000 118:S104-S114). Although, in monotherapy ribavirin reduces serum amino transferase levels to normal in 40% of patients, it does not lower serum levels of HCV-RNA. Ribavirin also exhibits significant toxicity and is known to induce anemia. Viramidine is a ribavirin prodrug converted ribavirin by adenosine deaminase to in hepatocytes. (J. Z. Wu, Antivir. Chem. Chemother. 2006 17(1):33-9)

Interferons (IFNs) have been available for the treatment of chronic hepatitis for nearly a decade. IFNs are glycoproteins produced by immune cells in response to viral infection. Two distinct types of interferon are recognized: Type 1 includes several interferon alphas and one interferon beta, type 2 includes interferon gamma. Type 1 interferons are produced mainly by infected cells and protect neighboring cells from de novo infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary. Cessation of therapy results in a 70% relapse rate and only 10-15% exhibit a sustained virological response with normal serum alanine transferase levels. (Davis, Luke-Bakaar, supra)

One limitation of early IFN therapy was rapid clearance of the protein from the blood. Chemical derivatization of IFN with polyethyleneglycol (PEG) has resulted in proteins with substantially improved pharmacokinetic properties. PEGASYS® is a conjugate interferon α −2a and a 40 kD branched mono-methoxy PEG and PEG-INTRON® is a conjugate of interferon α −2b and a 12 kD mono-methoxy PEG. (B. A. Luxon et al., Clin. Therap. 2002 24(9):13631383; A. Kozlowski and J. M. Harris, J. Control. Release 2001 72:217-224).

Combination therapy of HCV with ribavirin and interferon-α currently is the optimal therapy for HCV. Combining ribavirin and PEG-IFN (infra) results in a sustained viral response (SVR) in 54-56% of patients with type 1 HCV. The SVR approaches 80% for type 2 and 3 HCV. (Walker, supra) Unfortunately, combination therapy also produces side effects which pose clinical challenges. Depression, flu-like symptoms and skin reactions are associated with subcutaneous IFN-α and hemolytic anemia is associated with sustained treatment with ribavirin.

A number of potential molecular targets for drug development as anti-HCV therapeutics have now been identified including, but not limited to, the NS2-NS3 autoprotease, the NS3 protease, the NS3 helicase and the NS5B polymerase. The RNA-dependent RNA polymerase is absolutely essential for replication of the single-stranded, positive sense, RNA genome. This enzyme has elicited significant interest among medicinal chemists.

Nucleoside inhibitors can act either as a chain terminator or as a competitive inhibitor that interferes with nucleotide binding to the polymerase. To function as a chain terminator the nucleoside analog must be taken up by the cell in vivo and be converted in vivo to its triphosphate form to compete as a substrate at the polymerase nucleotide binding site. This conversion to the triphosphate is commonly mediated by cellular kinases which impart additional structural limitations on any nucleoside. In addition this requirement for phosphorylation limits the direct evaluation of nucleosides as inhibitors of HCV replication to cell-based assays (J. A. Martin et al., U.S. Pat. No. 6,846,810; C. Pierra et al., J. Med. Chem. 2006 49(22):6614-6620; J. W. Tomassini et al., Antimicrob. Agents and Chemother. 2005 49(5):2050; J. L. Clark et al., J. Med. Chem. 2005 48(17):2005).

Compounds of the present invention and their isomeric forms and pharmaceutically acceptable salts thereof are also useful in treating viral infections, in particular, hepatitis C infection, and diseases in living hosts when used in combination with each other and with other biologically active agents, including but not limited to the group consisting of interferon, a pegylated interferon, ribavirin, protease inhibitors, polymerase inhibitors, small interfering RNA compounds, antisense compounds, nucleotide analogs, nucleoside analogs, immunoglobulins, immunomodulators, hepatoprotectants, anti-inflammatory agents, antibiotics, antivirals and antiinfective compounds. Such combination therapy may also comprise providing a compound of the invention either concurrently or sequentially with other medicinal agents or potentiators, such as ribavirin and related compounds, amantadine and related compounds, various interferons such as, for example, interferon-alpha, interferon-beta, interferon gamma and the like, as well as alternate forms of interferons such as pegylated interferons. Additionally combinations of ribavirin and interferon, may be administered as an additional combination therapy with at least one of the compounds of the present invention.

Other forms of interferon α, as well as interferon β, γ, τ and ω are currently in clinical development for the treatment of HCV. For example, INFERGEN® (interferon alphacon-1) by InterMune, OMNIFERON® (natural interferon) by Viragen, ALBUFERON® by Human Genome Sciences, REBIF® (interferon β-1a) by Ares-Serono, Omega Interferon by BioMedicine, Oral Interferon Alpha by Amarillo Biosciences, and interferon γ, interferon τ, and interferon γ-1b by InterMune are in development.

HCV polymerase inhibitors are another target for drug discovery and compounds in development include R-1626, R-7128, IDX184/IDX102, PF-868554 (Pfizer), VCH-759 (ViroChem), GS-9190 (Gilead), A-837093 and A-848837 (Abbot), MK-3281 (Merck), GSK949614 and GSK625433 (Glaxo), ANA598 (Anadys), VBY 708 (ViroBay).

Inhibitors of the HCV NS3 protease also have been identified as potentially useful for treatment of HCV. Protease inhibitors in clinical trials include VX-950 (Telaprevir, Vertex), SCHSO3034 (Broceprevir, Schering), TMC435350 (Tibotec/Medivir) and ITMN-191 (Intermune). Other protease inhibitors in earlier stages of development include MK7009 (Merck), BMS-790052 (Bristol Myers Squibb), VBY-376 (Virobay), IDXSCA/IDXSCB (Idenix), BI12202 (Boehringer), VX-500 (Vertex), PHX1766 Phenomix).

Other targets for anti-HCV therapy under investigation include cyclophilin inhibitors which inhibit RNA binding to NS5b, nitazoxanide, Celgosivir (Migenix), an inhibitor of α-glucosidase-1, caspase inhibitors, Toll-like receptor agonists and immunostimulants such as Zadaxin (SciClone).

### SUMMARY OF THE INVENTION

There is currently no preventive treatment of Hepatitis C virus (HCV) and currently approved therapies, which exist only against HCV, are limited. Design and development of new pharmaceutical compounds is essential.

The present invention provides a compound according to formula I, or a pharmaceutically acceptable salt thereof, wherein:

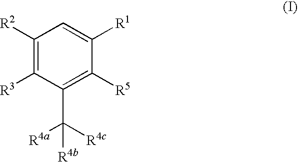

R¹ is selected from the group consisting of A-1, A-2, A-3 or A-4 wherein the dotted line is either a single or a double bond.

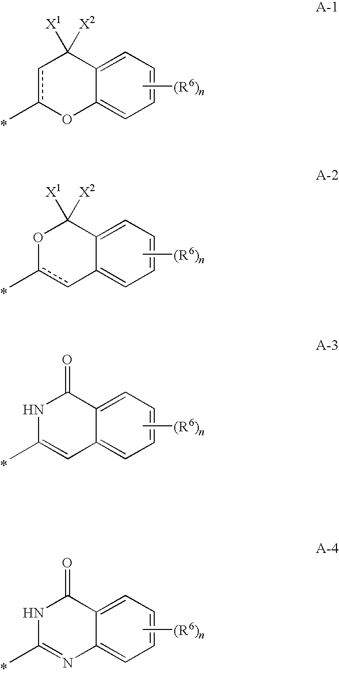

X¹ and X² each are hydrogen or X¹ and X² together are oxo.

R² is a heteroaryl radical selected from the group consisting of 2-oxo-1,2-dihydro-pyridin-3-yl, 3-oxo-3,4-dihydro-pyrazin-2-yl, 3-oxo-2,3-dihydro-pyridazin-4-yl, 2-oxo-1,2-dihydro-pyrimidin-4-one-5-yl and 6-oxo-1,6-dihydro-[1,2,4]triazin-5-yl said heteroaryl being optionally substituted by halogen, C₁₋₆ alkyl, C₁₋₃ haloalkyl, C₁₋₆ alkoxy, optionally substituted aryl-C₁₋₃ alkyl, —X—(CH₂)ₘNR^{c}R^{d} or X—(CH₂)ₘCO₂H wherein X is oxygen or a bond, m is 1 to 5 and R^{c} and R^{d} are independently hydrogen or C₁₋₃ alkyl or R^{c} and R^{d} together with the nitrogen atom to which they are attached are a cyclic amine.

R³ is hydrogen, fluorine or R³ and R^{4a} together are CH₂—O and together with atoms to which they are attached form a 2,3-dihydrobenzofuran or an indane.

R^{4a}, R^{4b} and R^{4c} (i) when taken independently are selected independently from C₁₋₃ alkyl, C₁₋₂ alkoxy, C₁₋₂ fluoroalkyl, C₁₋₃ hydroxyalkyl, cyano or hydroxy or (ii) when taken together, R^{4a} and R^{4b} together are C₂₋₄ alkylene and R^{4c} is hydrogen, C₁₋₃ alkyl, C₁₋₂ alkoxy, halogen, C₁₋₃ hydroxyalkyl, cyano or C₁₋₂ fluoroalkyl or R^{4a} and R^{4b} together with the carbon to which they are attached are 3-oxetanyl, or tetrahydrofuran-2-yl or (iii) either R⁵ or R³ and R^{4a} together are CH₂—O or (CH₂)₂ and together with atoms to which they are attached form a 2,3-dihydro-benzofuran or an indane and R^{4b} and R^{4c} are C₁₋₃ alkyl or (iv) R^{4a}, R^{4b} and R^{4c}; along with the carbon to which they are attached are a cyclopropyl, trifluoromethyl or 2,2,2-trifluoroethyl group.

R⁵ is hydrogen, C₁₋₆ alkyl, C₁₋₆ haloalkyl, C₁₋₆ alkoxy, C₁₋₆ haloalkoxy, C₁₋₃ alkoxy-C₁₋₆ alkoxy, halogen or R⁵ and R^{4a} together are CH₂—O and together with atoms to which they are attached form a 2,3-dihydrobenzofuran or an indane.

R⁶ is halogen, C₁₋₃ acylamino-C₁₋₆ alkyl, (CH₂)ₙNR^{a}R^{b} or (CH₂)ₙCONR^{a}R^{b}.

R^{a} and R^{b} are independently in each occurrence hydrogen, C₁₋₆ alkyl, C₁₋₃ haloalkyl, C₁₋₆ acyl, C₁₋₆ alkylsulfonyl, C₁₋₆ haloalkylsulfonyl, C₃₋₇ cycloalkylsulfonyl, C₃₋₇cycloalkyl-C₁₋₃ alkyl-sulfonyl, C₁₋₆ alkoxy-C₁₋₆ alkylsulfonyl or (CH₂)₁₋₃NR^{e}R^{f} wherein R^{e} and R^{f} are independently hydrogen or C₁₋₆ alkyl or R^{e} and R^{f} together with the nitrogen to which they are attached are an optionally substituted cyclic amine.

n is independently in each occurrence zero to two.

A pharmaceutically acceptable salt of a compound according to formula I.

The present invention also provides a method for treating a disease a Hepatitis C Virus (HCV) virus infection by administering a therapeutically effective quantity of a compound according to formula I to a patient in need thereof. The compound can be administered alone or co-administered with other antiviral compounds or immunomodulators.

The present invention also provides a method for inhibiting replication of HCV in a cell by administering a compound according to formula I in an amount effective to inhibit HCV.

The present invention also provides a pharmaceutical composition comprising a compound according to formula I and at least one pharmaceutically acceptable carrier, diluent or excipient.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “- - - - - -” drawn through a bond each refer to the point of attachment of a functional group or other chemical moiety to the rest of the molecule of which it is a part. Thus, for example:

MeC(═O)OR⁴ wherein

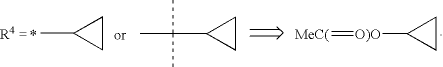

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

It will be appreciated by the skilled artisan that some of the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g. enantiomers), racemic mixtures or partially resolved mixtures of the compounds of formulae I and, where appropriate, the individual tautomeric forms thereof.

The racemates can be used as such or can be resolved into their individual isomers. The resolution can afford stereochemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively individual isomers can be separated chemically from a mixture by forming diastereomeric salts with a chiral acid, such as the individual enantiomers of 10-camphorsulfonic acid, camphoric acid, .alpha.-bromocamphoric acid, tartaric acid, diacetyltartaric acid, malic acid, pyrrolidone-5-carboxylic acid, and the like, fractionally crystallizing the salts, and then freeing one or both of the resolved bases, optionally repeating the process, so as obtain either or both substantially free of the other; i.e., in a form having an optical purity of >95%. Alternatively the racemates can be covalently linked to a chiral compound (auxiliary) to produce diastereomers which can be separated by chromatography or by fractional crystallization after which time the chiral auxiliary is chemically removed to afford the pure enantiomers.

The compounds of formula I may contain a basic center and suitable acid addition salts are formed from acids which form non-toxic salts. Examples of salts of inorganic acids include the hydrochloride, hydrobromide, hydroiodide, chloride, bromide, iodide, sulfate, bisulfate, nitrate, phosphate, hydrogen phosphate. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulfate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts see Berge et al, J. Pharm. Sci., 1977 66:1-19 and G. S. Paulekuhn et al. J. Med. Chem. 2007 50:6665.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001). The starting materials and reagents used in preparing these compounds generally are either available from commercial suppliers, such as Aldrich Chemical Co., or are prepared by methods known to those skilled in the art following procedures set forth in references. Materials, reagents and the like to which reference are made in the following description and examples are obtainable from commercial sources, unless otherwise noted. General synthetic procedures have been described in treatise such as Fieser and Fieser's Reagents for Organic Synthesis; Wiley & Sons: New York, Volumes 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40 and will be familiar to those skilled in the art.

In an embodiment of the present invention there is provided a compound according to formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², m and n are as described hereinabove.

In another embodiment of the present invention there is provided a compound according to formula I wherein R² is a heteroaryl radical selected from the group consisting of 2-oxo-1,2-dihydro-pyridin-3-yl, 3-oxo-3,4-dihydro-pyrazin-2-yl, 3-oxo-2,3-dihydro-pyridazin-4-yl, 2-oxo-1,2-dihydro-pyrimidin-4-one-5-yl and 6-oxo-1,6-dihydro-[1,2,4]triazin-5-yl said heteroaryl being optionally substituted by halogen, C₁₋₆ alkyl, C₁₋₃ C₁₋₆ alkoxy; R^{4a}, R^{4b} and R^{4c} (i) when taken independently are selected independently from C₁₋₃ alkyl, C₁₋₂ alkoxy, C₁₋₂ fluoroalkyl, C₁₋₃ hydroxyalkyl, cyano or hydroxy or (ii) when taken together, R^{4a} and R^{4b} together are C₂₋₄ alkylene and R^{4c} is hydrogen, C₁₋₃ alkyl, C₁₋₂ alkoxy, halogen, C₁₋₃ hydroxyalkyl, cyano or C₁₋₂ fluoroalkyl or R^{4a} and R^{4b} together with the carbon to which they are attached are 3-oxetanyl, or tetrahydrofuran-2-yl or (iii) either R⁵ or R³ and R^{4a} together are CH₂—O or (CH₂)₂ and together with atoms to which they are attached form a 2,3-dihydro-benzofuran or an indane and R^{4b} and R^{4c} are C₁₋₃ alkyl and R¹, R², R³, R^{4a}, R^{4b}, R^{4c}, R⁵, R⁶, R^{a}, R^{b}, X¹, X² and n are as defined herein above.

In another embodiment of the present invention there is provided a compound according to formula I wherein R¹ is a moiety of formula II-a, R³ is hydrogen and R⁵ is hydrogen or C₁₋₆ alkoxy.

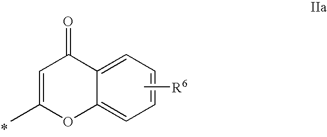

In another embodiment of the present invention there is provided a compound according to formula I wherein R¹ is a moiety of formula II-a, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl.

In a another embodiment of the present invention there is provided a compound according to formula I wherein R¹ is a moiety of formula II-a, R³ is hydrogen, R⁵ is hydrogen or C₁₋₆ alkoxy and R^{4a} and R^{4b} together are C₂ alkylene and R^{4c} is cyano, chloro C₁₋₃ fluoroalkyl.

In another embodiment of the present invention there is provided a compound according to formula I wherein R¹ is a moiety of formula II-b, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl

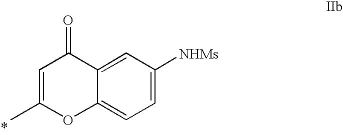

In another embodiment of the present invention there is provided a compound according to formula I wherein R¹ is a moiety of formula II-c, R³ is hydrogen and R⁵ is hydrogen or C₁₋₆ alkoxy.

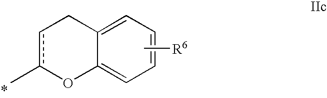

In another embodiment of the present invention there is provided a compound according to formula I wherein R¹ is a moiety of formula II-d, R³ is hydrogen, and R⁵ is hydrogen or C₁₋₆ alkoxy.

In another embodiment of the present invention there is provided a compound according to formula I wherein R¹ is a moiety of formula II-k, R³ is hydrogen, and R⁵ is hydrogen or C₁₋₆ alkoxy.

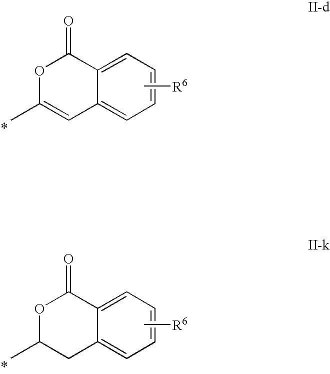

In another embodiment of the present invention there is provided a compound according to formula I wherein R¹ is a moiety of formula II-d, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl.

In another embodiment of the present invention there is provided a compound according to formula I wherein R¹ is a moiety of formula II-d, R³ is hydrogen, R⁵ is hydrogen or C₁₋₆ alkoxy and R^{4a} and R^{4b} together are C₂ alkylene and R^{4c} is cyano, chloro C₁₋₃ fluoroalkyl.

In another embodiment of the present invention there is provided a compound according to formula I wherein R¹ is a moiety of formula II-k, R³ is hydrogen, R⁵ is hydrogen or C₁₋₆ alkoxy and R^{4a} and R^{4b} together are C₂ alkylene and R^{4c} is cyano, chloro C₁₋₃ fluoroalkyl.

In another embodiment of the present invention there is provided a compound according to formula I wherein R¹ is a moiety of formula II-e, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl

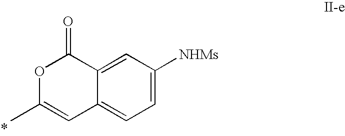

In another embodiment of the present invention there is provided a compound according to formula I wherein R¹ is a moiety of formula II-f, R³ is hydrogen and R⁵ is hydrogen or C₁₋₆ alkoxy.

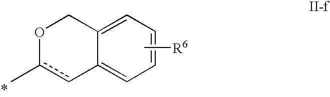

In another embodiment of the present invention there is provided a compound according to formula I wherein R¹ is a moiety of formula II-f, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl.

In still another embodiment of the present invention there is provided a compound according to formula I wherein R¹ is a moiety of formula II-f, R³ is hydrogen, R⁵ is hydrogen or C₁₋₆ alkoxy and R^{4a} and R^{4b} together are C₂ alkylene and R^{4c} is chloro, cyano or C₁₋₃.

In another embodiment of the present invention there is provided a compound according to formula I wherein R¹ is a moiety of formula II-g, R³ is hydrogen and R⁵ is hydrogen or C₁₋₆ alkoxy.

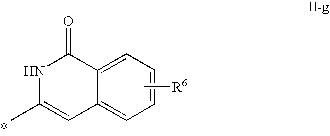

In another embodiment of the present invention there is provided a compound according to formula I wherein R¹ is a moiety of formula II-g, R³ is hydrogen R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl.

In still another embodiment of the present invention there is provided a compound according to formula I wherein R¹ is a moiety of formula II-g, R³ is hydrogen, R⁵ is hydrogen or C₁₋₆ alkoxy and R^{4a} and R^{4b} together are C₂ alkylene and R^{4c} is chloro, cyano or C₁₋₃.

In another embodiment of the present invention there is provided a compound according to formula I wherein R¹ is a moiety of formula II-h, R³ is hydrogen R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl

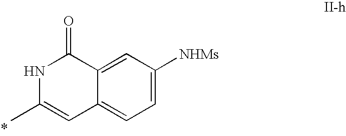

In another embodiment of the present invention there is provided a compound according to formula I wherein R¹ is a moiety of formula II-h, R³ is hydrogen and R⁵ is hydrogen or C₁₋₆ alkoxy.

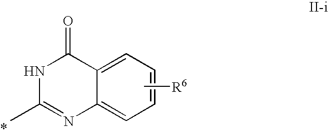

In another embodiment of the present invention there is provided a compound according to formula I wherein R¹ is a moiety of formula II-i, R³ is hydrogen R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl.

In another embodiment of the present invention there is provided a compound according to formula I wherein R¹ is a moiety of formula II-j, R³ is hydrogen, R⁵ is hydrogen or C₁₋₆ alkoxy and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl.

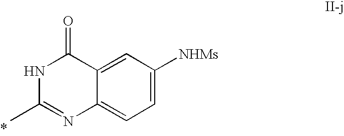

In another embodiment of the present invention there is provided a compound selected from I-1 to I-14 of TABLE 1.

In another embodiment of the present invention there is provide a method of treating a HCV infection in a patient in need thereof comprising administering a therapeutically effective amount of a compound according to formula I wherein R¹, R², R³, R^{4a}, R^{4b}, R^{4e}, R⁵, R⁶, R^{a}, R^{b}, R^{c}, R^{d}, R^{e}, R^{f}, X, X¹, X², m and n are as defined hereinabove.

In another embodiment of the present invention there is provide a method of treating a HCV infection in a patient in need thereof comprising co-administering a therapeutically effective amount of a compound according to formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², m and n are as defined herein above and at least one immune system modulator and/or at least one antiviral agent that inhibits replication of HCV.

In another embodiment of the present invention there is provide a method of treating a disease caused by HCV in a patient in need thereof comprising co-administering a therapeutically effective amount of a compound according to formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², m and n are as defined herein above and at least one immune system modulator selected from interferon, interleukin, tumor necrosis factor or colony stimulating factor.

In another embodiment of the present invention there is provide a method of treating a HCV infection in a patient in need thereof comprising co-administering a therapeutically effective amount of a compound according to formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², m and n are as defined herein above and an interferon or chemically derivatized interferon.

In another embodiment of the present invention there is provide a method of treating a HCV infection in a patient in need thereof comprising co-administering a therapeutically effective amount of a compound according to formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², m and n are as defined herein above and another antiviral compound selected from the group consisting of a HCV protease inhibitor, another HCV polymerase inhibitor, a HCV helicase inhibitor, a HCV primase inhibitor and a HCV fusion inhibitor.

In another embodiment of the present invention there is provided a method for inhibiting viral replication in a cell by delivering a therapeutically effective amount of a compound of the formula I wherein R¹, R², R³, R^{4a}, R^{4b}, R^{4c}, R⁵, R⁶, R^{a}, R^{b}, R^{c}, R^{d}, R^{e}, R^{f}, X, X¹, X², X², m and n are as defined herein above admixed with at least one pharmaceutically acceptable carrier, diluent or excipient.

In another embodiment of the present invention there is provided a composition comprising a compound according to formula A-R wherein R¹, R², R³, R^{4a}, R^{4b}, R^{4c}, R⁵, R⁶, R^{a}, R^{b}, R^{c}, R^{d}, R^{e}, R^{f}, X, X¹, X², X², m and n are as defined herein above admixed with at least one pharmaceutically acceptable carrier, diluent or excipient.

The term “alkyl” as used herein without further limitation alone or in combination with other groups, denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₆ alkyl” as used herein refers to an alkyl composed of 1 to 6 carbons. Examples of alkyl groups include, but are not limited to, lower alkyl groups include methyl, ethyl, propyl, iso-propyl, n-butyl, tert-butyl, tert-butyl, neopentyl, hexyl, and octyl.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (hetero)aryl refers to either an aryl or a heteroaryl group.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. C₀₋₄ alkylene refers to a linear or branched saturated divalent hydrocarbon radical comprising 1-4 carbon atoms or, in the case of Cₒ, the alkylene radical is omitted. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, 1-fluoroethyl, 1-chloroethyl, 12-fluoroethyl, 2-chloroethyl, 2-bromoethyl, 2,2-dichloroethyl, 3-bromopropyl or 2,2,2-trifluoroethyl. The term “fluoroalkyl” as used herein refers to a haloalkyl moiety wherein fluorine is the halogen.

The term “haloalkoxy” as used herein refers to a group —OR where R is haloalkyl as defined herein. The term “haloalkylthio” as used herein refers to a group —SR where R is haloalkyl as defined herein.

The term “halogen” or “halo” as used herein means fluorine, chlorine, bromine, or iodine.

The terms “hydroxyalkyl” and “alkoxyalkyl” as used herein denotes alkyl radical as herein defined wherein one to three hydrogen atoms on different carbon atoms is/are replaced by hydroxyl or alkoxy groups respectively. A C₁₋₃ alkoxy-C₁₋₆ alkyl moiety refers to a C₁₋₆ alkyl substituent in which 1 to 3 hydrogen atoms are replaced by a C₁₋₃ alkoxy and the point of attachment of the alkoxy is the oxygen atom.

The terms “hydroxyalkoxy” and “alkoxyalkoxyl” as used herein denotes alkoxy radical as herein defined wherein one to three hydrogen atoms on different carbon atoms is/are replaced by hydroxyl or alkoxy groups respectively. A C₁₋₃ alkoxy-C₁₋₆ alkoxy moiety refers to a C₁₋₆ alkoxy substituent in which 1 to 3 hydrogen atoms are replaced by a C₁₋₃ alkoxy and the point of attachment of the alkoxy is the oxygen atom.

The terms “alkoxycarbonyl” and “aryloxycarbonyl” as used herein denotes a group of formula —C(═O)OR wherein R is alkyl or aryl respectively and alkyl and aryl are as defined herein.

The term “cyano” as used herein refers to a carbon linked to a nitrogen by a triple bond, i.e., —C≡N. The term “nitro” as used herein refers to a group —NO₂. The term “carboxy” as used herein refers to a group —CO₂H.

The term oxo refers to a doubly bonded oxygen (═O), i.e. a carbonyl group.

The term “acyl” (or “alkanoyl”) as used herein denotes a group of formula —C(═O)R wherein R is hydrogen or lower alkyl as defined herein. The term or “alkylcarbonyl” as used herein denotes a group of formula —C(═O)R wherein R is alkyl as defined herein. The term C₁₋₆ acyl or “alkanoyl” refers to a group —C(═O)R contain 1 to 6 carbon atoms. The Cᵢ acyl group is the formyl group wherein R═H and a C6 acyl group refers to hexanoyl when the alkyl chain is unbranched. The term “arylcarbonyl” or “aroyl” as used herein means a group of formula C(═O)R wherein R is an aryl group; the term “benzoyl” as used herein an “arylcarbonyl” or “aroyl” group wherein R is phenyl.

The term “acylamino” as used herein denotes a group of formula —NHC(═O)R wherein R is hydrogen or lower alkyl as defined herein. C₁₋₆ acyl-amino refers to an acylamino group wherein the C(═O)R moiety contains a total of 6 carbon atoms.

The term “cyclic amine” as used herein refers to a saturated carbon ring, containing from 3 to 6 carbon atoms as defined above, and wherein at least one of the carbon atoms is replaced by a heteroatom selected from the group consisting of N, O and S, for example, piperidine, piperazine, morpholine, thiomorpholine, di-oxo-thiomorpholine, pyrrolidine, pyrazoline, imidazolidine, azetidine wherein the cyclic carbon atoms are optionally substituted by one or more substituents, selected from the group consisting of halogen, hydroxy, phenyl, lower alkyl, lower alkoxy or 2-hydrogen atoms on a carbon are both replace by oxo (═O). When the cyclic amine is a piperazine, one nitrogen atom can be optionally substituted by C₁₋₆ alkyl, C₁₋₆ acyl, C₁₋₆ alkylsulfonyl.

The terms “alkylsulfonyl” and “arylsulfonyl” as used herein denotes a group of formula —S(═O)₂R wherein R is alkyl or aryl respectively and alkyl and aryl are as defined herein. The term C₁₋₃ alkylsulfonylamido as used herein refers to a group RSO₂NH— wherein R is a C₁₋₃ alkyl group as defined herein. The terms C₁₋₆ haloalkylsulfonyl, C₃₋₇ cycloalkylsulfonyl, C₃₋₇ cycloalkyl-C₁₋₃ alkyl-sulfonyl or C₁₋₆ alkoxy-C₁₋₆ alkylsulfonyl refer to a compound, S(═O)₂R wherein R is C₁₋₆ haloalkyl, C₃₋₇ cycloalkyl, C₃₋₇ cycloalkyl-C₁₋₃ alkyl and C₁₋₆ alkoxy-C₁₋₆ alkyl, respectively.

The terms “alkylsulfonylamino” and “arylsulfonylamino” as used herein denotes a group of formula —NR′S(═O)₂R wherein R is alkyl or aryl respectively, R′ is hydrogen or C₁₋₃ alkyl, and alkyl and aryl are as defined herein.

The term “sulfamoyl” as used herein refers to the radical —S(O)₂NH₂. The terms “N-alkylsulfamoyl” and “N,N-dialkylsulfamoyl” as used herein refers to the radical —S(O)₂NR′R″, wherein R′ and R″ are hydrogen and lower alkyl and R′ and R″ are independently lower alkyl respectively. Examples of N-alkylsulfamoyl substituents include, but are not limited to methylaminosulfonyl, iso-propylaminosulfonyl. Examples of N,N-dialkylsulfamoyl substituents include, but are not limited to dimethylaminosulfonyl, iso-propyl-methylaminosulfonyl.

The term “carbamoyl” as used herein means the radical —CONH₂. The prefix “N-alkylcabamoyl” and “N,N-dialkylcarbamoyl” means a radical CONHR' or CONR′R″ respectively wherein the R′ and R″ groups are independently alkyl as defined herein. The prefix N-arylcabamoyl” denotes the radical CONHR' wherein R′ is an aryl radical as defined herein.

The term “arylalkyl” or “aralkyl” as used herein denotes the radical R′R″-, wherein R′ is an aryl radical as defined herein, and R″ is an alkylene radical as defined herein with the understanding that the attachment point of the arylalkyl moiety will be on the alkylene radical. “Optionally substituted aryl-C₁₋₃ alkyl” refers to a compound where the alkylene chain is 1 to 3 carbons and the aryl may substituted. The term “benzyl” as used herein refers to a C₆H₅CH₂ radical. Optional substitution includes hydroxy, thio, cyano, C₁₋₆ alkyl, C₁₋₆ alkoxy, C₁₋₆ haloalkyl, C₁₋₆ haloalkoxy, halogen unless otherwise indicated.

The term “aryl” as used herein refers to a phenyl ring. Optionally substituted aryl by substituted by includes hydroxy, thio, cyano, C₁₋₆ alkyl, C₁₋₆ alkoxy, C₁₋₆ haloalkyl, C₁₋₆ haloalkoxy, halogen unless otherwise indicated.

The term “pyridine” (“pyridinyl) refers to a six-membered heteroaromatic ring with one nitrogen atom. The terms “pyrimidine” (pyrimidinyl), “pyrazine” (“pyrazinyl”) and “pyridazine” (“pyridazinyl”) refer to a six-membered nonfused heteroaromatic ring with two nitrogen atoms disposed in a 1,3, a 1,4 and a 1,2 relationship respectively. The respective radical names are in parentheses.

To avoid ambiguity, the following nomenclature and numbering systems are used: chromen-4-one (A), 4H-chromene (B), chroman (C), isochromene (D), 1H-isochromen-1-one (E), isochroman (F), 2H-isoquinolin-1-one (G) and 3,4-dihydro-isoquinolin-1-one (F).

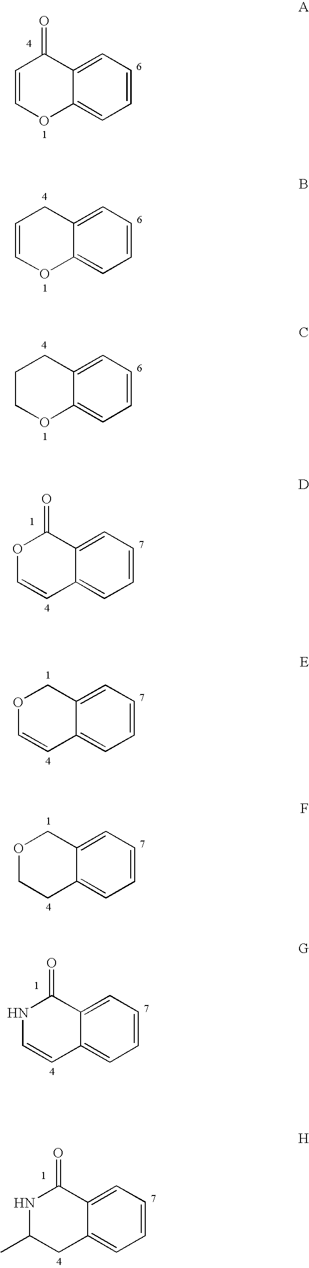

The terms (i) 3-oxo-3,4-dihydro-pyrazin-2-yl, (ii) 3-oxo-2,3-dihydro-pyridazin-4-yl or (iii) 2-oxo-1,2-dihydro-pyrimidin-4-one-5-yl and (iv) 2-oxo-1,2-dihydro-pyridin-3-yl and (v) 6-oxo-1,6-dihydro-[1,2,4]triazin-5-yl refer to the following moieties:

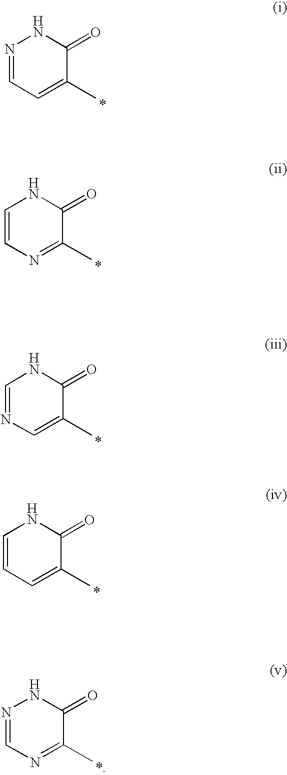

The phrase “substituted at least by (CH₂)ₙNR^{c}R^{d}” in reference to Ar simply indicates the ring is substituted by (CH₂)ₙNR^{c}R^{d} but other additional optional substitutions within the scope of the claim are permitted.

Compounds of the present invention and their isomeric forms and pharmaceutically acceptable salts thereof are also useful in treating viral infections, in particular, hepatitis C infection, and diseases in living hosts when used in combination with each other and with other biologically active agents, including but not limited to the group consisting of interferon, a pegylated interferon, ribavirin, protease inhibitors, polymerase inhibitors, small interfering RNA compounds, antisense compounds, nucleotide analogs, nucleoside analogs, immunoglobulins, immunomodulators, hepatoprotectants, anti-inflammatory agents, antibiotics, antivirals and anti-infective compounds. Such combination therapy may also comprise providing a compound of the invention either concurrently or sequentially with other medicinal agents or potentiators, such as ribavirin and related compounds, amantadine and related compounds, various interferons such as, for example, interferon-alpha, interferon-beta, interferon gamma and the like, as well as alternate forms of interferons such as pegylated interferons. Additionally combinations of ribavirin and interferon, may be administered as an additional combination therapy with at least one of the compounds of the present invention.

In one embodiment, the compounds of the present invention according to formula I are used in combination with other active therapeutic ingredients or agents to treat patients with an HCV viral infection. According to the present invention, the active therapeutic ingredient used in combination with the compound of the present invention can be any agent having a therapeutic effect when used in combination with the compound of the present invention. For example, the active agent used in combination with the compound of the present invention can be interferons, ribavirin analogs, HCV NS3 protease inhibitors, nucleoside inhibitors of HCV polymerase, non-nucleoside inhibitors of HCV polymerase, NS5A inhibitors, and other drugs for treating HCV, or mixtures thereof.

Examples of the nucleoside NS5b polymerase inhibitors include, but are not limited to NM-283, valopicitabine, R1626, PSI-6130 (R1656), IDX184 and IDX102 (Idenix) BILB 1941.

Examples of the non-nucleoside NS5b polymerase inhibitors include, but are not limited to HCV-796 (ViroPharma and Wyeth), MK-0608, MK-3281 (Merck), NM-107, R7128 (R4048), VCH-759, GSK625433 and GSK625433 (Glaxo), PF-868554 (Pfizer), GS-9190 (Gilead), A-837093 and A848837 (Abbot Laboratories), ANA598 (Anadys Pharmaceuticals), GL100597 (GNLB/NVS), VBY 708 (ViroBay), benzimidazole derivatives (H. Hashimoto et al. WO 01/47833, H. Hashimoto et al. WO 03/000254, P. L. Beaulieu et al. WO 03/020240 A2; P. L. Beaulieu et al. U.S. Pat. No. 6,448,281 B1; P. L. Beaulieu et al. WO 03/007945 A1), benzo-1,2,4-thiadiazine derivatives (D. Dhanak et al. WO 01/85172 A1, filed May 10, 2001; D. Chai et al., WO2002098424, filed Jun. 7, 2002, D. Dhanak et al. WO 03/037262 A2, filed Oct. 28, 2002; K. J. Duffy et al. WO03/099801 A1, filed May 23, 2003, M. G. Darcy et al. WO2003059356, filed Oct. 28, 2002; D. Chai et al. WO 2004052312, filed Jun. 24, 2004, D. Chai et al. WO2004052313, filed Dec. 13, 2003; D. M. Fitch et al., WO2004058150, filed Dec. 11, 2003; D. K. Hutchinson et al. WO2005019191, filed Aug. 19, 2004; J. K. Pratt et al. WO 2004/041818 A1, filed Oct. 31, 2003), 1,1-dioxo-4H-benzo[1,4]thiazin-3-yl derivatives (J. F. Blake et al. in U.S. Patent Publication US20060252785 and 1,1-dioxo-benzo[d]isothazol-3-yl compounds (J. F. Blake et al. in U.S. Patent Publication 2006040927).

Examples of the HCV NS3 protease inhibitors include, but are not limited to SCH-503034 (Schering, SCH-7), VX-950 (telaprevir, Vertex), BILN-2065 (Boehringer-Ingelheim, BMS-605339 (Bristol Myers Squibb), and ITMN-191 (Intermune).

Ribavirin analogs and the ribavirin prodrug viramidine (taribavirin) have been administered with interferons to control HCV.

Commonly used abbreviations include: acetyl (Ac), aqueous (aq.), atmospheres (Atm), 2,2′-bis(diphenylphosphino)-1,1′-binaphthyl (BINAP), tert-butoxycarbonyl (Boc), di-tent-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), methanol (MeOH), melting point (mp), MeSO₂— (mesyl or Ms), methyl (Me), acetonitrile (MeCN), m-chloroperbenzoic acid (MCPBA), mass spectrum (ms), methyl tent-butyl ether (MTBE), N-methylmorpholine (NMM), N-methylpyrrolidone (NMP), phenyl (Ph), propyl (Pr), iso-propyl (i-Pr), pounds per square inch (psi), pyridine (pyr), room temperature (rt or RT), satd. (saturated), tert-butyldimethylsilyl or t-BuMe₂Si (TBDMS), triethylamine (TEA or Et₃N), triflate or CF₃SO₂— (Tf), trifluoroacetic acid (TFA), O-benzotriazol-1-yl-N,N,N′,N′-tetramethyluronium tetrafluoroborate (TBTU), thin layer chromatography (TLC), tetrahydrofuran (THF), tetramethylethylenediamine (TMEDA), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n-), iso (i-), secondary (sec-), tertiary (tent-) and neo- have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds encompassed by the present invention and within the scope of the invention are provided in TABLE I. These examples and preparations which follow are provided to enable those skilled in the art to more clearly understand and to practice the present invention. They should not be considered as limiting the scope of the invention, but merely as being illustrative and representative thereof. Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. I-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Some compounds in following schemes are depicted as a Markush structure with generalized substituents; however, one skilled in the art will immediately appreciate that the nature of the R groups as defined in the claims can varied as defined in the appended claims to afford the various compounds contemplated in this invention. Moreover, the reaction conditions are exemplary and alternative conditions can be identified without undue experimentation. The reaction sequences in the following examples are not meant to limit the scope of the invention as set forth in the claims.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

TABLE I

| Cpd | 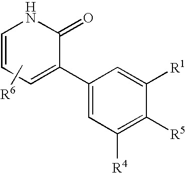 | | | | | | |
|---|---|---|---|---|---|---|---|
| # | R¹ | R⁵ | R4 | R⁶ | IC₅₀¹ | mp | ms |
| I-1 | 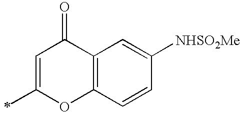 | OMe | *—CMe₃ | H | 0.003 | >300 | 495 |
| I-2 | 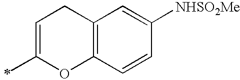 | OMe | *—CMe₃ | H | 0.014 | | 481 |
| I-3 | 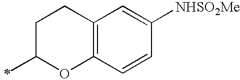 | OMe | *—CMe₃ | H | 0.001 | | 482 |
| I-4 | 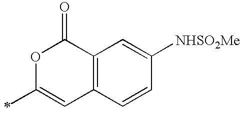 | —OMe | *—CMe₃ | H | 0.004 | 293.0- 295.0 | 495 |
| I-5 | 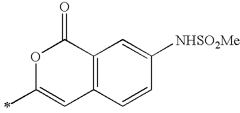 | H | *—CMe₃ | H | 0.009 | >300 | 465 |
| I-6 | 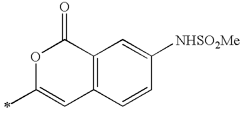 | —OMe | *—CMe₃ | 5-F | 0.003 | 295.0- 297.0 | 513 |
| I-7 | 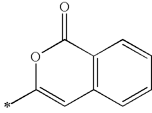 | —OMe | *—CMe₃ | H | 0.002 | 253.0- 255.0 | 402 |
| I-8 | 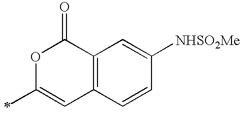 | —OMe | *—CMe₃ | 6- OMe | | 231.0- 233.0 | 525 |
| I-9 | 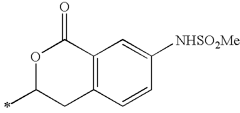 | —OMe | *—CMe₃ | H | 0.006 | 235 .0- 237.0 | 497 |
| I-10 | 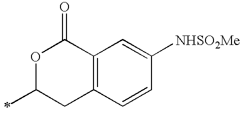 | —OMe | *—CMe₃ | H | 0.001 | 170.0- 175.0 | 483 |
| I-11 | 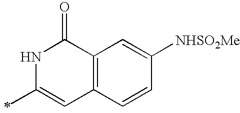 | —OMe | *—CMe₃ | H | 0.004 | 252.0- 254.0 | 494 |
| I-12 | 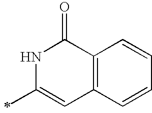 | —OMe | *—CMe₃ | H | 0 .003 | 138.0- 140.0 | 401 |
| I-13 | 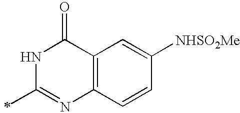 | —OMe | *—CMe₃ | H | 0.009 | 228.0- 230.0 | 495 |

| | | | | |
|---|---|---|---|---|
| I-14 | 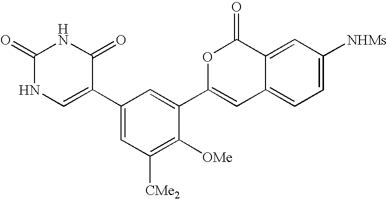 | 0.001 | >300 | 512 |
| I-15 | 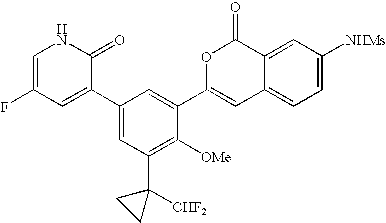 | 0.0002 | | 547 |

1. IC₅₀ NS5B Polymerase Assay (μM), see example 11

Compounds of the present invention with an isochromene substituent on the phenyl ring were prepared by a gold-catalyzed cycloisomerization of acetylenic acids and esters (E. Genin et al., J. Am. Chem. Soc. 2006 128(10):3112-3113). Methyl 2-phenylethynyl-benzoates undergo AuCl₃-mediated 6-endo cyclization to directly afford the desired isochromen-1-ones A-1. (E. Marchal et al., Tetrahedron 2007 63:9979-9990) Thus, the AuCl₃ catalyzed cyclization of A-2 afforded A-1 which was debenzylated to afford the pyridone.

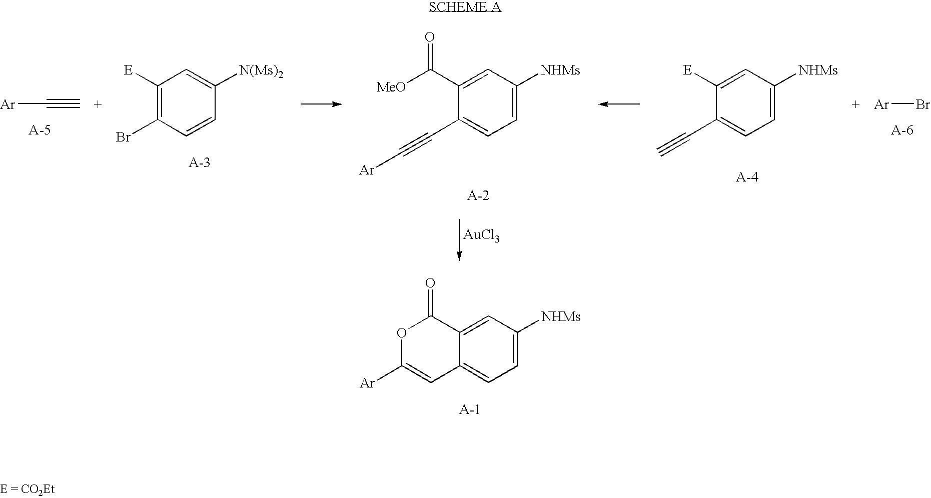

The requisite acetylenic esters were prepared by a Sonogashira coupling of a suitable substituted aryl halide A-6 and an optionally substituted alkyl ortho-ethynyl-benzoate (A-4). The aryl moiety in SCHEME A is a substituted 3-(3-halophenyl-phenyl)-1H-pyridin-2-one, 4-(3-halophenyl-phenyl)-2H-pyridazin-3-one, 3-(3-halophenyl-phenyl)-1H-pyrazin-2-one, 5-(3-halophenyl-phenyl)-3H-pyrimidin-4-one or 5-(3-halophenyl-phenyl)-1H-[1,2,4]triazin-6-one. As will be apparent to one skilled in the art, the acetylene can originate on either of the aryl residues.

The Sonogashira coupling (K. Sonogashira et al., Tetrahedron Lett. 1975 4467-4470; K. Sonogashira, Comprehensive Organic Synthesis; B. M. Trost and I. Fleming Eds.; Pergamon Press, Oxford, 1991; Vol. 3, Chapter 2.4, p 521) is typically carried out in the presence of a palladium catalyst such as Pd(PPh₃)₄ or Pd(II) Cl₂(PPh₃)₂ and a cuprous salt, for example CuI, a dialkyl- or trialkylamine such as diethylamine, diisopropylamine, TEA and the like at temperature from RT to 100° C. The reaction can be carried out using the amine base as the solvent or with other organic solvents including hydrocarbons, ethers, alcohols, aqueous DMA and the like.

Compounds encompassed in the present claims comprising an 1H-1-isochromen-1-one or isochroman substituted aryl moiety can be prepared by modification of the isochromene. Hydrolysis of the A-1 lactone affords a keto-acid which can be reduced to the hydroxy-acid and relactonized (see, e.g., Example 6). The isochroman ring can be prepared by reduction of the isochromene to the diol which can be recyclized under acidic conditions to afford the corresponding isochroman (see, e.g., Example 8).

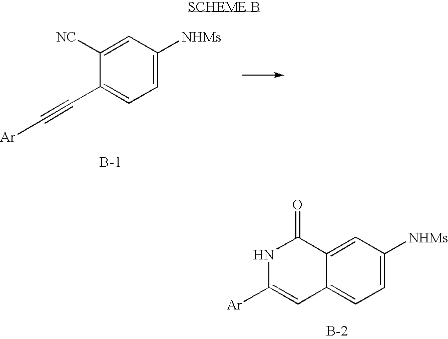

Compounds encompassed in the present claims wherein the aryl ring is substituted by a 2H-isoquinolin-1-one were prepared by a related intramolecular cyclization of the corresponding cyano-acetylene (SCHEME B) Hydrolysis of the nitrile (B-1) in the presence of hydrido(dimethylphosphinous acid-kP)[hydrogen bis(dimethylphosphnito-kP]platinum (II) (CASRN 173416-05-2; X-b Jiang et al., Platinum-Catalyzed Selective Hydration of Hindered Nitriles and Nitriles with Acid- or Base Sensitive Groups, J. Org. Chem. 2004 69(7):2327-31; T. Ghaffar and A. W. Parkins, A New Homogenous Platinum Containing Catalyst for the Hydrolysis of Nitriles. Tetrahedron Lett. 1995 36(47):8657-8660), induced an intramolecular cycloisomerization to afford the desired 2H-isoquinolin-1-ones B-2 occurred.

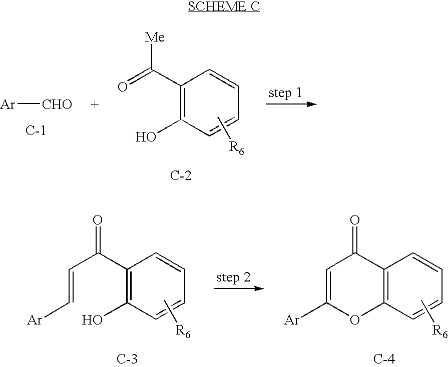

Compounds encompassed in the present claims wherein the aryl ring is substituted by a chromen-4-one can be prepared by an Aldol condensation of a benzaldehyde C-1 and an ortho-hydroxy-acetophenone C-2 to afford a β-aryl vinyl ketone which can undergo intramolecular cyclization and subsequent dehydrohalogenation to afford the chromen-4-one C-4 when contacted with iodine (SCHEME C). (M. Cabrera et al., Bioorg. Med. Chem. 2007 15:3356-3367). Reduction of the ketone to afford a 4H-chromene was carried out by treating the chromen-4-one with lithium aluminum hydride. (T. G. C. Baird et al., J. Chem. Soc. Perkin Trans. 11983 1831) Further reduction of the olefin to afford the chroman ring can be carried out using by catalytic hydrogenation (see, e.g., Example 3). One skilled in the art will appreciate that further substitution of C-2 is easily accomplished which will afford substituted derivatives

The requisite aldehydes are readily prepared from ortho-alkyl-phenols such as 2-tert-butyl phenol by formylation to afford 3-tent-butyl-2-hydroxy-benzaldehyde which can be O-alkylated and brominated to afford 5-bromo-3-tert-butyl-2-methoxy-benzaldehyde. The bromine substituent allow for the ready introduction of the heteroaryl rings encompassed by R² in the claimed compounds utilizing palladium-catalyze cross coupling reactions. The aldehyde C-1 can be expeditiously conveniently converted to the alkynes utilized in SCHEME A (A-5) condensing C-1 with (1-diazo-2-oxo-propyl)-phosphonic acid diethyl ester. (R. Muller et al. Syn Lett 1996 6:521). 3-tent-Butyl-5-hydroxy-benzaldehyde can also be utilized analogously by converting the phenol to a triflate which can be subjected to palladium catalyzed cross-coupling reactions.

Aryl acetylenes (A-5) are also accessible from 1-alkyl-3,5-dibromobenzenes such as 1,3-dibromo-5-tert-butylbenzene (CASRN 19316-09-2) which can be subjected to sequential palladium catalyzed couplings to introduce the acetylene and the requisite heteroaryl substituents. Another useful precursor is 3,5-dibromo-benzoacetonitrile which cane be modified to incorporate cyclopropyl substitution onto the aryl ring.

Quinazolines typified by I-13 are similarly accessible from C-1 by condensation with ortho-amino-benzamide derivatives as exemplified in Example 13.

### Anti-Viral Activity

The activity of the inventive compounds as inhibitors of HCV activity may be measured by any of the suitable methods known to those skilled in the art, including in vivo and in vitro assays. For example, the HCV NS5B inhibitory activity of the compounds of formula I can determined using standard assay procedures described in Behrens et al., EMBO J. 1996 15:12-22, Lohmann et al., Virology 1998 249:108-118 and Ranjith-Kumar et al., J. Virology 2001 75:8615-8623. Unless otherwise noted, the compounds of this invention have demonstrated in vitro HCV NS5B inhibitory activity in such standard assays. The HCV polymerase assay conditions used for compounds of the present invention are described in Example 8. Cell-based replicon systems for HCV have been developed, in which the nonstructural proteins stably replicate subgenomic viral RNA in Huh7 cells (V. Lohmann et al., Science 1999 285:110 and K. J. Blight et al., Science 2000 290:1972. The cell-based replicon assay conditions used for compounds of the present invention are described in Example 4. In the absence of a purified, functional HCV replicase consisting of viral non-structural and host proteins, our understanding of Flaviviridae RNA synthesis comes from studies using active recombinant RNA-dependent RNA-polymerases and validation of these studies in the HCV replicon system. Inhibition of recombinant purified HCV polymerase with compounds in vitro biochemical assays may be validated using the replicon system whereby the polymerase exists within a replicase complex, associated with other viral and cellular polypeptides in appropriate stoichiometry. Demonstration of cell-based inhibition of HCV replication may be more predictive of in vivo function than demonstration of HCV NS5B inhibitory activity in vitro biochemical assays.

### Dosage and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate. The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent such as ribavirin, a nucleoside HCV polymerase inhibitor, another HCV non-nucleoside polymerase inhibitor or HCV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

Furthermore, the term “treatment” of a HCV infection, as used herein, also includes treatment of a disease or a condition associated with or mediated by HCV infection, or the clinical symptoms thereof.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

A therapeutically effective amount of a compound of the present invention, and optionally one or more additional antiviral agents, is an amount effective to reduce the viral load or achieve a sustained viral response to therapy. Useful indicators for a sustained response, in addition to the viral load include, but are not limited to liver fibrosis, elevation in serum transaminase levels and necroinflammatory activity in the liver. One common example, which is intended to be exemplary and not limiting, of a marker is serum alanine transminase (ALT) which is measured by standard clinical assays. In some embodiments of the invention an effective treatment regimen is one which reduces ALT levels to less than about 45 IU/mL serum.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### Example 1

### N-{2-[3-tert-Butyl-2-methoxy-5-(2-oxo-1,2-dihydro-pyridin-3-yl)-phenyl]-4-oxo-4H-chromen-6-yl}-methanesulfonamide (I-1)

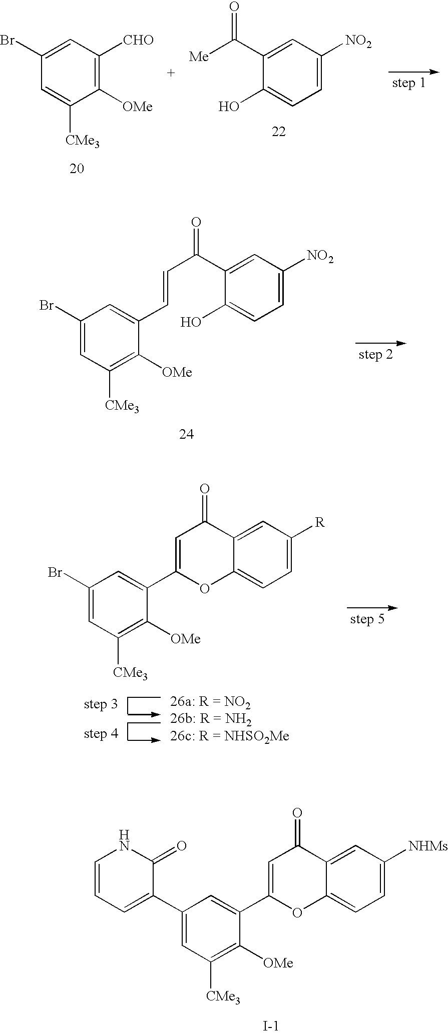

### 5-bromo-3-tert-butyl-2-methoxy-benzaldehyde (20)

step a—To a solution of 3-tent-butyl-2-hydroxybenzaldehyde (CASRN 24623-65-2, 5.00 g) and DCM (20 mL) at 0° C. was added dropwise a solution of Br₂ (1.45 mL) in DCM (15 mL) over a period of 30 min. After the addition was complete the reaction was stirred for 1 h before the organic volatiles were removed under reduced pressure to afford 7.23 g of 5-bromo-3-tent-butyl-2-hydroxybenzaldehyde (21) as a light yellowish solid.

step b—A mixture of 21 (3.83 g), MeI (2.32 mL) and K₂CO₃ (6.18 g) in DMF (50 mL) was heated at 50° C. for 1 h then cooled to RT and diluted with ether and water. The organic layer was thrice washed with water then brine, dried (MgSO₄) and concentrated to afford 3.99 g of 20 as a yellow solid.

2-oxo-1,2-dihydropyridine-3-boronic acid (28)—To a solution of 3-bromo-2-oxo-1,2-dihydropyridine (3.3 g, 19 mmol) in THF (200 mL) cooled to −76° C. was added dropwise over 15 min TMEDA (6.5 g, 56 mmol), followed by n-butyllithium (2.5M in hexane, 58 mmol). The resulting mixture was stirred for 15 min at −76° C. and then warmed to RT. Upon reaching an internal temperature of 19° C., the reaction mixture was cooled to 0° C., and B(OMe)₃ (4.0 g, 39 mmol) was added dropwise over 15 min. After the addition was complete, the reaction mixture was warmed to RT and was stirred for 15 h. The mixture was then cooled to 0° C. and a small amount of ice was added followed by 2M aqueous HCl (100 mL). The THF was removed under reduced pressure, and the aqueous solution was washed twice with DCM. Concentrated aqueous NaOH was added slowly until pH 5 was attained and a precipitate formed. The mixture was cooled to 0° C. and stirred for 10 min. The solid was collected by filtration, washed with cold water, and dried under vacuum to afford 1.83 g (69%) of 112 as a yellow solid

step 1—To a solution of 20 (2.00 g, 7.38 mmol), 22 (1.34 g, 7.40 mmol) in EtOH (15 mL), was added freshly powdered KOH (0.51 g, 9.11 mmol). The reaction was stirred overnight at RT and then heated at reflux for another day. The reaction mixture was concentrated and diluted with EtOAc. 6N HCl (2 mL) was added and a yellow precipitate formed. The suspension was concentrated, suspended in water and filtered to give 3.12 g (98%) of 24 as an orange solid.

step 2—A solution of 24 (0.50 g, 1.15 mmol), iodine (33.9 mg, 0.133 mmol) in DMSO (6 mL) was heated at reflux for 1.5 h. The reaction mixture was cooled to RT and poured into ice water. The resulting precipitate was filtered and dried to afford 487 mg of a tan solid. The filtrate was extracted with EtOAc and the extract washed with brine. The organic extract was dried (Na₂SO₄) and concentrated to afford 46 mg of a tan oil which was identical to the precipitate and afford a combined yield of 533 mg (100%) of 26a.

step 3—To a solution of 26a (533 mg, 1.237 mmol) in EtOAc (10 mL) and DMF (10 mL), was added 5 nCl₂.2H₂O (1.12 g, 4.964 mmol). The resulting suspension was stirred overnight at RT and then cooled to 0° C. and quenched with aq. NaHCO₃. The resulting suspension was filtered through CELITE. The filtrate was thrice washed with brine, dried (Na₂SO₄), filtered and concentrated. The crude product was purified by SiO₂ chromatography eluting with 30% EtOAc/hexane to afford 215 mg (43%) of 26b as an orange solid.

step 4—To a solution of 26b (215 mg, 0.536 mmol) in DCM (15 mL) at 0° C., was added pyridine (0.130 mL, 1.607 mmol) and methanesulfonyl chloride (0.080 mL, 1.029 mmol). The reaction was gradually warmed to RT and stirred overnight. The solution was diluted with DCM, washed sequentially with sat'd. CuSO₄, twice with 1N HCl, dried (Na₂SO₄), filtered and concentrated. The crude product was purified by SiO₂ chromatography eluting with an EtOAc/hexane gradient (20% to 30% EtOAc) to afford 26c.

step 5—A microwave tube was charged with 26c (36 mg, 0.075 mmol), 28 (16 mg, 0.115 mmol), Pd(PPh₃)₄ (8.9 mg, 0.008 mmol), Na₂CO₃ (25 mg, 0.236 mmol) and a mixture of MeOH (3 mL) and DCM (1 mL), sealed and irradiated in a microwave reactor at 115° C. for 30 min. The reaction mixture was concentrated, diluted with EtOAc, washed with water, dried (Na₂SO₄), filtered and concentrated. The crude product was purified on a preparative SiO₂ TLC plate developed with 3:1 hexane/EtOAc to afford 13.5 mg (36%) of I-1 as an off-white solid.

### Example 2

### N-{2-[3-tert-Butyl-2-methoxy-5-(2-oxo-1,2-dihydro-pyridin-3-yl)-phenyl]-4H-chromen-6-yl}-methanesulfonamide (I-2)

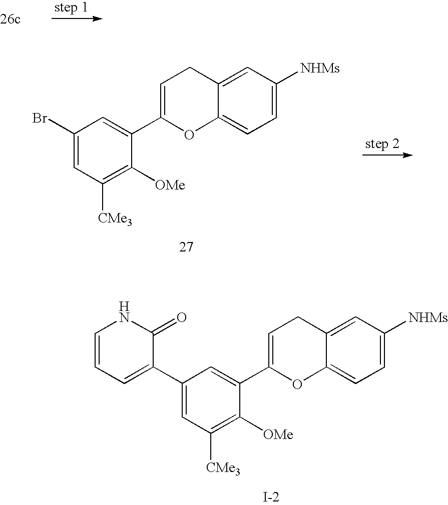

step 1—To a solution of 26c (100 mg, 0.209 mmol) in THF (5 mL) cooled to 0° C., was added LiALH₄ (0.420 mL, 0.420 mmol, 1.0 M THF solution). The reaction mixture was gradually warmed to RT over 1.5 h, then cooled to 0° C., and quenched with aq. NaHCO₃ (1 mL). The suspension was diluted with EtOAc, washed with brine, dried (Na₂SO₄), filtered and concentrated. The crude residue was purified by SiO₂ chromatography eluting with an EtOAc/hexane gradient (20% to 30% EtOAc) to give 40 mg (41%) of N-[2-(5-bromo-3-tert-butyl-2-methoxy-phenyl)-4H-chromen-6-yl]-methane sulfonamide (27) as an orange oil.

step 2—Palladium catalyzed cross-coupling of 27 and 28 was carried in accord with the procedure in step 5 of Example 1. The crude product was purified on a preparative SiO₂ TLC plate developed with 2:1 hexane/EtOAc to afford 12 mg of a yellow solid which was further purified by HPLC to give 4.1 mg (10%) of I-2a pale yellow solid.

### Example 3

### N-{2-[3-tert-Butyl-2-methoxy-5-(2-oxo-1,2-dihydro-pyridin-3-yl)-phenyl]-chroman-6-yl}-methanesulfonamide (I-3)

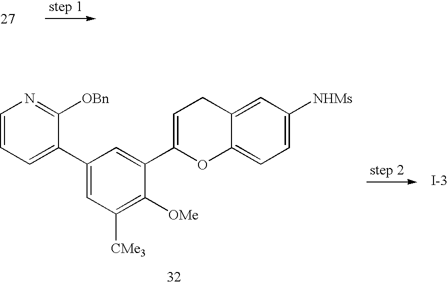

2-benzyloxy-pyridin-3-yl boronic acid (30)—A solution of 2-benzyloxy-3-bromo-pyridine (2.50 gq, 9.47 mmol), Pd(II) Cl₂(PPh₃)₂ (232 mg, 0.28 mmol), KOAc (2.32 g, 23.67 mmol), bis-(pinacolato)diborane (2.95 g, 11.36 mmol) and DME (75 mL) was heated at 70° C. for 26 h. The reaction mixture was cooled and partitioned between Et₂O and water. The organic phase was separated, dried and evaporated. The crude product was purified by SiO₂ chromatography eluting with an EtOAc/hexane gradient (0 to 5% EtOAc) to afford 1.81 g of 2-benzyloxy-pyridin-3-yl boronic acid containing a small amount of bis-(pinacolato)diborane.

step 1—A sealed tube containing 27 (75 mg, 0.156 mmol), 30 (53 mg, 0.231 mmol), Pd(PPh₃)₄ (19 mg, 0.016 mmol), Na₂CO₃ (43 mg, 0.406 mmol) in a mixture of MeOH (3 mL) and DCM (1 mL) was irradiated in a microwave reactor at 115° C. for 30 min. The reaction mixture was concentrated, diluted with EtOAc, washed with brine, dried (Na₂SO₄), filtered and concentrated. The crude product was purified by SiO₂ chromatography eluting with an EtOAc/hexane gradient (10% to 30% EtOAc) to afford 36 mg (40%) of 32 as a colorless oil (40%).

step 2—To a solution of 32 (36 mg, 0.063 mmol) in EtOAc (5 mL) and MeOH (5 mL) was added palladium hydroxide (20% on carbon, 20 mg, 0.029 mmol). The reaction was stirred under an atmosphere of hydrogen overnight. The reaction mixture was filtered, concentrated, and purified on a preparative SiO₂ TLC plate developed with 2:1 EtOAc/hexane to afford 10.6 mg (35%) of I-3 as a white solid.

### Example 4

### N-{3-[3-tert-Butyl-2-methoxy-5-(2-oxo-1,2-dihydro-pyridin-3-yl)-phenyl]-1-oxo-1H-isochromen-7-yl}-methanesulfonamide (I-4)

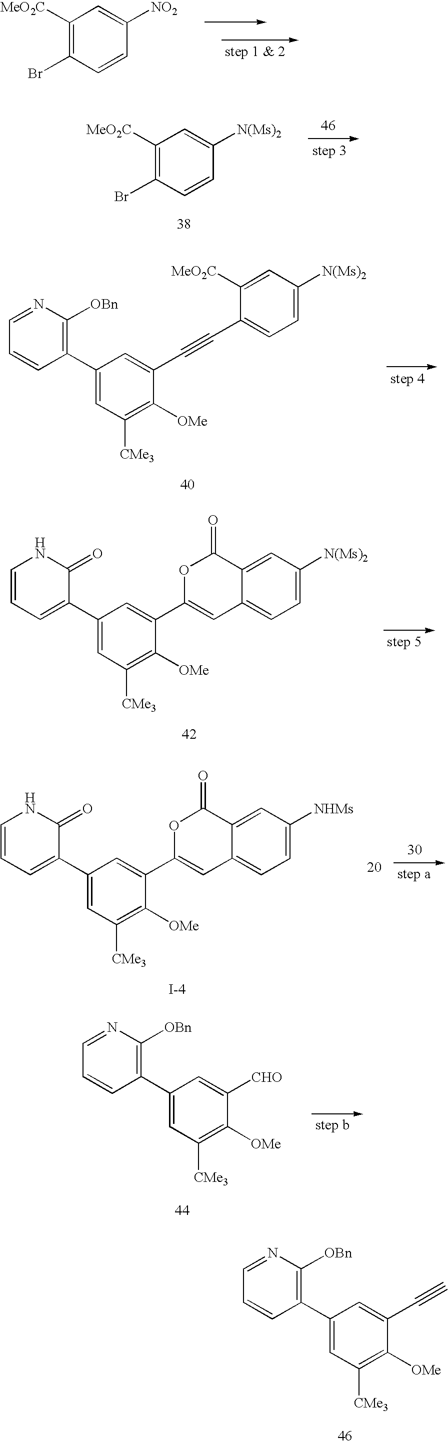

### 2-Benzyloxy-3-(3-tert-butyl-5-ethynyl-4-methoxy-phenyl)-pyridine (46)

step a—A sealed tube containing 20 (3.99 g, 14.72 mmol), 30 (5.07 g, 22.14 mmol), Pd(PPh₃)₄, (1.32 g, 1.142 mmol), Na₂CO₃ (3.93 g, 37.08 mmol) in a mixture of MeOH (33 mL) and DCM (9 mL) was irradiated in a microwave synthesizer at 115° C. for 30 min. The reaction mixture was concentrated, diluted with EtOAc, washed with brine, and dried (Na₂SO₄), filtered and evaporated. The crude product was purified by SiO₂ chromatography eluting with an EtOAc/hexane gradient (0 to 10% EtOAc) to afford 5.506 g (99%) of 5-(2-benzyloxy-pyridin-3-yl)-3-tert-butyl-2-methoxy-benzaldehyde 44 as an orange oil, which solidified on standing.

step b—To a solution of 44 (1.00 g, 2.667 mmol) in MeOH (20 mL) at −78° C., was added sodium methoxide (0.5M in MeOH, 11 mL, 5.5 mmol). A solution of dimethyl 1-diazo-2-oxopropylphoshonate (712 mg, 4.00 mmol) in MeOH (10 mL) was added dropwise and the resulting white suspension was gradually warmed to RT and stirred overnight. The reaction was quenched with saturated NaHCO₃ solution and concentrated. The crude residue was diluted with EtOAc, washed sequentially with saturated NaHCO₃, water, brine, dried (Na₂SO₄), filtered and evaporated. The crude product was purified by SiO₂ chromatography eluting with an EtOAc/hexane gradient (0 to 4% EtOAc) to afford 679 mg (69%) of 46 as a colorless oil step 1—To a mixture of methyl 2-bromo-5-nitro-benzoate (1.5 g, 5.8 mmol) and NH₄Cl (3.1 g, 58 mmol) in MeOH (50 mL) and H₂O (25 mL) heated to 70° C. was added iron powder (1.62 g, 29 mmol) over a period of 60 min. After addition was completed, stirring was continued for 45 min, and then the reaction mixture was cooled, filtered through CELITE and the pad was washed with MeOH. The filtrate was concentrated and partitioned between H₂O and EtOAc. The organic phase was washed with brine, dried (MgSO₄), filtered and concentrated to afford 1.34 g (100%) of methyl 2-bromo-5-amino-benzoate (48).

step 2—To a solution of 48 (1.34 g, 5.8 mmol) in DCM (30 mL) cooled to 5° C. was added TEA (4.04 ml, 2.52 g, 29 mmol), followed by dropwise addition of a solution of methanesulfonyl chloride (1.08 mL, 1.6 g, 14 mmol) in DCM (10 mL) over a period of 15 min. The reaction mixture was stirred at RT overnight, quenched with aqueous 1N HCl, and extracted with EtOAc. The combined extracts were washed with brine, dried (MgSO₄), filtered and concentrated to afford 4.5 g (100%) of 38.

step 3—To a solution of 46 (163 mg, 0.44 mmol) in DMF (4 mL) was added CuI (2.1 mg, 0.01 mmol) and PPh₃ (3.0 mg, 0.01 mmol). This solution mixture was purged with argon for 5 min and then PdCl₂(PPh₃)₂ (15.4 mg, 0.02 mmol) was added followed by 38 (212 mg, 0.55 mmol) and DIPEA (100 μL, 71 mg, 0.55 mmol). Argon was bubbled through the solution then the reaction mixture was heated to 75° C. for 6 h. The mixture was cooled, quenched with aq. 1N HCl and twice extracted EtOAc. The combined extracts were washed with water and brine, dried (MgSO₄), filtered and concentrated. The crude material was purified by SiO₂ chromatography eluting with an EtOAc/hexane gradient (10% to 75% EtOAc) to afford 240 mg (80%) of 40

step 4—To a solution of 40 (120 mg, 0.18 mmol) in TFA (1.0 mL) was added AuCl₃ (3 mg). Argon was bubbled through the solution for 3 minutes. The tube was sealed and irradiated in a microwave reactor at 110° C. for 30 min. The reaction mixture was partitioned between H₂O and EtOAc. The organic solution was washed with brine, dried (MgSO₄), filtered and concentrated. The crude material was purified by SiO₂ chromatography eluting with 5% MeOH/DCM to afford 37 mg (31%) of 42.

step 5—A mixture of 42 (31 mg, 0.54 mmol) and DIPEA (0.3 mL) in DMF (1.5 mL) was heated at 75° C. for 15 h. The mixture was cooled, diluted with EtOAc, washed sequentially with aq. 1N HCl, water and brine, dried (MgSO₄), filtered and concentrated. The crude purified was purified by SiO₂ chromatography eluting with 10% MeOH/DCM to afford 22 mg (81.5%) of I-4.

### Example 5

### N-{3-[3-tert-Butyl-5-(2-oxo-1,2-dihydro-pyridin-3-yl)-phenyl]-1-oxo-1H-isochromen-7-yl}-methanesulfonamide (I-5)

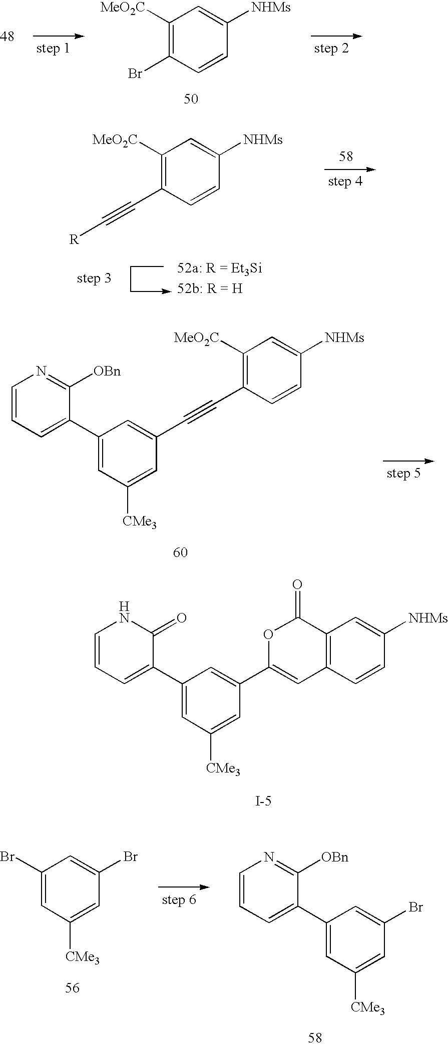

step 1—To a cooled (5° C.) solution of 48 (4.46 g, 19.4 mmol), pyridine (8 mL, 7.6 g, 97 mmol) in DCM (90 mL) was added dropwise over a period of 20 min a solution of methanesulfonyl chloride (1.65 ml, 2.43 g, 21.3 mmol) in DCM (10 mL). The reaction mixture was stirred at RT overnight and then poured into aq. 1N HCl solution. The resulting solution was extracted with EtOAc, washed with brine, dried (MgSO₄), filtered and concentrated to afford 5.7 g (95%) of 50.

step 2—To a solution of the (triethylsilyl)acetylene (630 mg, 4.5 mmol) in DMF (25 mL) was added CuI (57 mg, 0.3 mmol) and PPh₃ (420 mg, 0.06 mmol). This solution mixture was purged with argon for 5 min then PdCl₂(PPh₃)₂ (15.4 mg, 0.02 mmol) was added followed by 50 (1.16 g, 3.0 mmol) and TEA (12 mL). Argon was bubbled through the solution and the reaction mixture was heated at 75° C. for 6 h under an argon atmosphere. The mixture was cooled, quenched with aqueous 1N HCl and twice extracted with EtOAc. The organic solution was washed sequentially with H₂O and brine, dried (MgSO₄), filtered and concentrated. The crude material was purified by SiO₂ chromatography eluting with an EtOAc/hexane gradient (10% to 45% EtOAc) to afford 1.66 g (70%) of 52a.

step 3—To a solution of 52a (1.66 g, 4.5 mmol) in THF (75 mL) cooled to −30° C. was added dropwise, over a period of 15 min, a solution of tetrabutylammonium fluoride (5 mL, 1M solution in THF). The reaction mixture was stirred at RT overnight and poured into aq. sat'd. NH₄Cl solution. The resulting solution was extracted with EtOAc and the extracts washed with brine, dried (MgSO₄), filtered and concentrated. The crude material was purified by SiO₂ chromatography eluting with an EtOAc/hexane gradient (10% to 60% EtOAc) to afford 0.889 g (78%) of 52b.

step 4—A mixture of the 52b (100 mg, 0.4 mmol), 2-benzyloxy-3-(3-bromo-5-tert-butyl-phenyl)-pyridine (54, 230 mg, 0.6 mmol), CuI (3.7 mg, 0.02 mmol), PdCl₂(PPh₃)₂ (28 mg, 0.04 mmol), TEA (5 mL) in DMF (10 mL) was stirred at 70° C. for 2 h. The mixture was cooled, quenched with aq. 1N HCl and the resulting mixture was extracted twice with EtOAc. The organic solution was washed with sequentially water, brine, dried (MgSO₄), filtered and concentrated. The crude material was purified by SiO₂ chromatography eluting with an EtOAc/hexane gradient (10% to 70% EtOAc) to afford 39.2 mg (18%) of 60.

step 5—A tube was charged with 60 (77 mg, 0.136 mmol), AuCl₃ (3 mg) in TFA (1 mL), sealed and irradiated in a microwave reactor at 95° C. for 25 min. The mixture was concentrated and purified by SiO₂ chromatography eluting with an 30% acetone/DCM to afford 20 mg (32%) of I-5.

step 6—A tube was charged with 56 (2.50 g, 8.56 mmol), 30 (2.35 g, 10.27 mmol), Pd(PPh₃)₄ (0.494 g, 0.43 mmol), Na₂CO₃ (1.36 g, 12.84 mmol), MeOH (15 mL) and DCM (2 mL), sealed and irradiated in a microwave synthesizer at 115° C. for 30 min. The reaction mixture was concentrated and the crude product purified by SiO₂ chromatography eluting with a EtOAc/hexane gradient (1 to 10% EtOAc) to afford 3.78 g of 58 as a viscous colorless oil along 1.02 g of the bis-arylated byproduct.

### Example 6

### N-{3-[3-tert-Butyl-2-methoxy-5-(2-oxo-1,2-dihydro-pyridin-3-yl)-phenyl]-1-oxo-isochroman-7-yl}-methanesulfonamide (I-9)

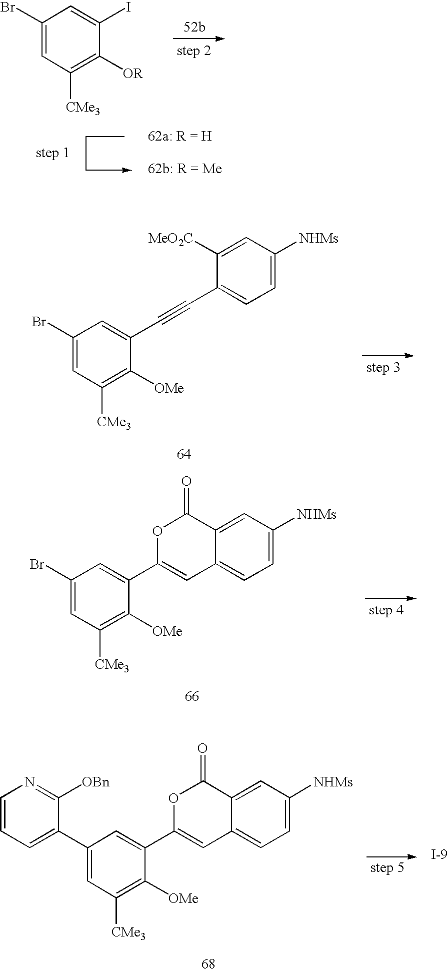

### 4-bromo-2-tent-butyl-6-iodo-phenol (62a)

To an ice-cold solution of 4-bromo-2-tert-butyl phenol (2.8 g, 86 wt %) dissolved in MeOH containing NaI (3.28 g) and NaOH (0.88 g) was added an aq. solution of NaOCl (4.5 wt %, 68.75 mL). The addition was continued until yellow color persisted (1.6 equivalents). To the resulting solution was added sat'd. aq. Na₂SO₃ (10 mL) and HOAc (2.5 mL) which resulted in the formation of a precipitate. The MeOH was evaporated and the residue suspended in H₂O (50 mL) and aged at 40° C. for 2 h. then slowly cooled to RT. The solid was filtered, washed with H₂O and dried in vacuo at 50° C. overnight to afford 6.74 g (87%) of 62a.

step 1—To a solution of 62a (4.40 g, 12.4 mmol), iodomethane (7.7 mL, 17.6 g, 124 mmol) in acetone (80 mL) was added K₂CO₃ (8.60 g, 62 mmol) and the resulting solution was stoppered and stirred overnight at RT. The reaction mixture was diluted with hexanes (100 mL) and the mixture was filtered over a plug of SiO₂. The filtrate was concentrated to afford 4.6 g (100%) of 62b.

step 2—A solution 52b (230 mg, 0.91 mmol), 62b (400 mg, 0.11 mmol), CuI (17 mg, 0.091 mmol), PdCl₂(PPh₃)₂ (130 mg, 0.18 mmol), TEA (5 mL) in DMF (10 mL) was stirred at 70° C. for 2 h. The mixture was cooled, quenched with aq. 1N HCl and twice extracted with EtOAc. The combined extracts were washed sequentially with H₂O and brine, dried (MgSO₄), filtered and concentrated. The crude material was purified by SiO₂ chromatography eluting with an EtOAc/hexane gradient (10% to 70% EtOAc) to afford 191 mg (42%) of 64.

step 3—A tube was charged with 64 (978 mg, 19.8 mmol), AuCl₃ (30 mg) and TFA (4 mL), sealed and irradiated in a microwave reactor at 100° C. for 45 min. The mixture was cooled and concentrated. The crude product was purified by SiO₂ chromatography eluting with an EtOAc/hexane gradient (10% to 60% EtOAc) to afford 0.95 g (100%) of 66.

step 4—A tube was charged with 66 (300 mg, 0.625 mmol), 30 (210 mg, 0.94 mmol), Na₂CO₃ (200 mg, 1.87 mmol) and Pd(PPh₃)₄ (72 mg, 0.0635 mmol) in MeOH (9 mL) and DCM (3 mL), sealed and irradiated in a microwave reactor at 100° C. for 30 min. The mixture was concentrated and purified by SiO₂ chromatography eluting with an EtOAc/hexane gradient (15% to 75% EtOAc) to afford 0.294 g (80.5%) of 68.

step 5—A solution of the 68 (120 mg, 0.21 mmol) in 5% KOH (4 mL) and EtOH (4 mL) was heated at reflux for 1.5 hours. The mixture was concentrated, diluted with H₂O and washed with Et₂O. The aqueous layer was acidified and extracted with EtOAc. The EtOAc solution was washed with brine, dried (MgSO₄), filtered and concentrated.

The residual yellow solid (keto-acid) was dissolved in EtOH (4 mL) and NaBH₄ (4.8 mg, 1.26 mmol) was added. The mixture was heated at for 4 h, cooled and partitioned between aqueous 1N HCl and EtOAc. The organic solution was washed with brine, dried (MgSO₄), filtered and concentrated.

The resulting hydroxy-acid was heated with acetic anhydride (1 mL) at 100° C. for 2 h, cooled and partitioned between H₂O and EtOAc. The EtOAc extract was washed sequentially with aq. sat'd. NaHCO₃ and brine, dried (MgSO₄), filtered and concentrated. The crude material was purified by SiO₂ chromatography eluting with 5% MeOH/DCM to afford 11.7 mg (12%) of I-9 along with 49.2 mg (42%) of N-{3-[3-(2-benzyloxy-pyridin-3-yl)-5-tent-butyl-phenyl]-1-oxo-isochroman-7-yl}-methanesulfonamide.

### Example 7

### 3-[3-tert-Butyl-4-methoxy-5-(1-oxo-1H-isochromen-3-yl)-phenyl]-1H-pyridin-2-one (I-7)

step 1—A stirred solution of 46 (300 mg, 0.81 mmol), methyl-2-iodobenzoate (250 mg, 0.97 mmol), CuI (8 mg, 0.04 mmol), PdCl₂(PPh₃)₂ (57 mg, 0.081 mmol), TEA (3 mL) in DMF (6 mL) was heated to 75° C. for 3 h. The mixture was cooled, quenched with aqueous 1N HCl and twice extracted with EtOAc. The combined extracts were washed sequentially with water and brine, dried (MgSO₄), filtered and concentrated. The crude material was purified by SiO₂ chromatography eluting with an EtOAc/hexane gradient (2% to 30% EtOAc) to afford 0.275 g (66%) of 2-[5-(2-benzyloxy-pyridin-3-yl)-3-tert-butyl-2-methoxy-phenylethynyl]-benzoic acid methyl ester (70).

step 2—A tube was charged with 70 (275 mg, 0.54 mmol), AuCl₃ (8 mg, 0.027 mmol)) and TFA (3 mL), sealed and irradiated in a microwave reactor at 100° C. for 30 min. The mixture was concentrated and purified by SiO₂ chromatography eluting with 2% MeOH/DCM to afford 84 mg (39%) of (I-7).

### Example 8

### N-{3-[3-tert-Butyl-2-methoxy-5-(2-oxo-1,2-dihydro-pyridin-3-yl)-phenyl]-isochroman-7-yl}-methanesulfonamide (I-10)

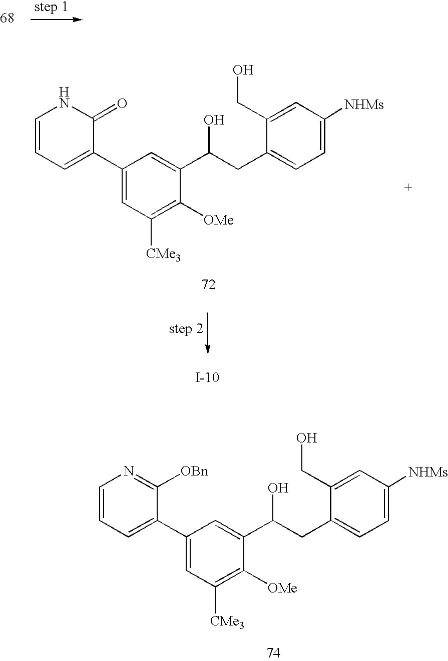

step 1—To a solution of 68 (49 mg, 0.084 mmol) in THF (5 mL) was added LiAlH₄ (126 mL, 1M in THF). The reaction mixture was heated at reflux for 2 h and then stirred overnight at RT. The reaction was quenched with 1N HCl and extracted with EtOAc. The combined extracts were washed with brine, dried (MgSO₄), filtered and concentrated. The crude material was purified by SiO₂ chromatography eluting with 6% MeOH/DCE to afford 72 (12.7 mg, 26%) and 74 (5.9 mg, 14%).

step 2—To a stirred solution of aq. 50% H₃PO₄ was added a solution of 72 (12.7 mg, 0.0215 mmol) dissolved in minimum volume of THF (1 mL) and the resulting solution was heated at 100° C. overnight. The reaction mixture was cooled and an ice-cold sat'd. aq. NaHCO₃ solution was cautiously added dropwise until the pH was approximately pH-6. The reaction was extracted with EtOAc and the combined extracts washed with brine, dried (MgSO₄), filtered and concentrated. The crude product was purified by SiO₂ chromatography eluting with 6% MeOH/DCM to afford 6 mg (10%) of I-10.

### Example 9

### N-{3-[3-tert-Butyl-2-methoxy-5-(6-methoxy-2-oxo-1,2-dihydro-pyridin-3-yl)-phenyl]-1-oxo-1H-isochromen-7-yl}-methanesulfonamide (I-8)

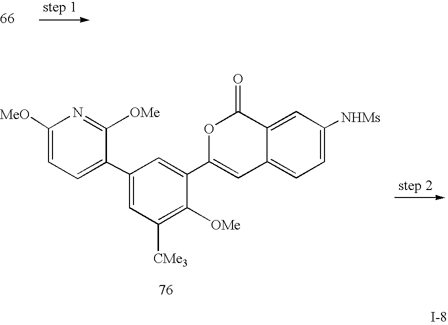

step 1—A tube was charged with 66 (50 mg, 0.104 mmol), B-(2,6-dimethoxy-3-pyridinyl)-boronic acid, (28 mg, 0.154 mmol, CASRN 221006-70-8), Na₂CO₃ (50 mg, 0.468 mmol) and Pd(PPh₃)₄ (12 mg, 0.01 mmol) in MeOH (3 mL) and DCM (1 mL), sealed and irradiated in a microwave reactor at 100° C. for 30 min. The mixture was concentrated and purified by SiO₂ chromatography eluting with 40% EtOAc/hexanes) to afford 42 mg (75%) of 76.

step 2—A reaction vessel charged with 76 (42 mg, 0.078 mmol), 48% HBr (0.1 mL) and HOAc (1 mL) was sealed and heated overnight at 65° C. The solution was neutralized with sat'd. aq. NaHCO₃ and extracted with EtOAc. The organic extracts were washed with brine, dried (MgSO₄), filtered and concentrated. The crude product was purified by SiO₂ chromatography eluting with 30% acetone/DCM to afford 10 mg (24%) of I-8.

### Example 10

### N-{3-[3-tert-Butyl-2-methoxy-5-(2-oxo-1,2-dihydro-pyridin-3-yl)-phenyl]-1-oxo-1,2-dihydro-isoquinolin-7-yl}-methanesulfonamide (I-11)

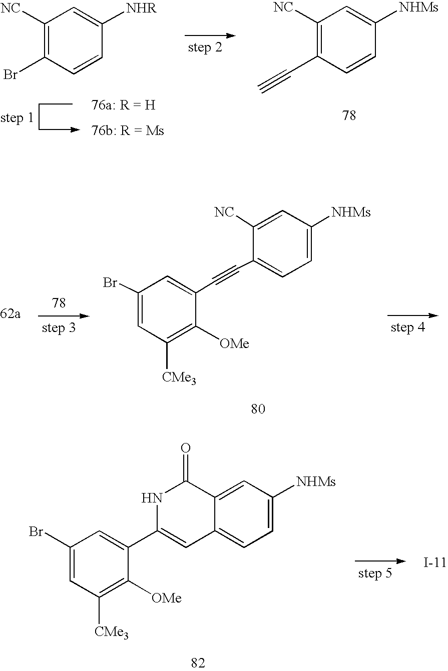

step 1—To a mixture of 76a (3.20 g, 16.24 mmol) in DCM (75 mL) cooled to 0° C. was added pyridine (1.51 mL, 19.49 mmol) and followed by MsCl (2.62 mL, 32.49 mmol). The resulting mixture was allowed to warm to RT and stirred for 24 h. The reaction was cooled to 0° C. and quenched with 1N aq. HCl solution. The reaction was concentrated and diluted with H₂O The resulting precipitate was filtered and washed with H₂O and dried in vacuo at 45° C. to afford 4.68 g of 69b.

step 2—Conversion of 76b to 78 was carried out in accord with the procedures in steps 2 and 3 of Example 5

step 3—A solution 78 (440 mg, 2.0 mmol), 62a (880 mg, 2.4 mmol), CuI (19 mg, 0.10 mmol), PdCl₂(PPh₃)₂ (140 mg, 0.20 mmol), TEA (10 mL) in DMF (20 mL) was stirred at 70° C. for 2 h. The mixture was cooled, quenched with aqueous 1N HCl and twice extracted with EtOAc. The organic extracts were washed sequentially with H₂O and brine, dried (MgSO₄), filtered and concentrated. The crude material was purified by SiO₂ chromatography eluting with an EtOAc/hexane gradient (10% to 70% EtOAc) to afford 468 mg (51%) of 80.

step 4—A solution of 80 (468 mg, 10 mmol) and hydrido(dimethylphosphinous acid-kP)platinum (81, 86 mg, 0.2 mmol, CASRN 173416-05-2)) in EtOH (40 mL) was heated at reflux for 2 h. The reaction mixture was cooled and concentrated. The crude material was purified by SiO₂ chromatography eluting with an EtOAc/hexane gradient (20% to 90% EtOAc) to afford 390 mg (81%) of 82.

step 5—A sealed tube containing the bromide 82 (70 mg, 0.15 mmol), 28 (30 mg, 0.22 mmol), Na₂CO₃ (46 mg, 0.44 mmol) and Pd(PPh₃)₄ (17 mg, 0.015 mmol) in MeOH (3 mL) and DCM (1 mL) was irradiated in a microwave reactor at 100° C. for 30 min. The mixture was concentrated and purified by SiO₂ chromatography eluting with 10% MeOH/DCM to afford 29.4 mg (40%) of I-11.

### Example 11

### N-{3-[3-tert-Butyl-5-(5-fluoro-2-oxo-1,2-dihydro-pyridin-3-yl)-2-methoxy-phenyl]-1-oxo-1H-isochromen-7-yl}-methanesulfonamide (I-6)

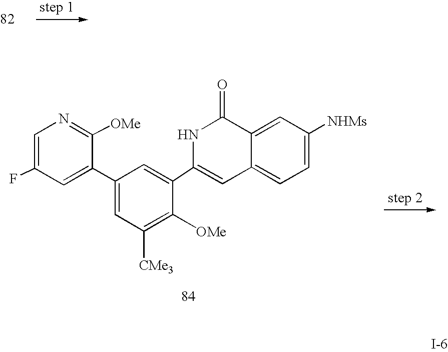

step 1—A vial was charged with 82 (80 mg, 0.17 mmol), 5-fluoro-2-methoxypyridine-3-boronic acid (31 mg, 0.18 mmol, CASRN 957120-32-0), Na₂CO₃, (0.50 mmol), Pd(PPh₃)₄ (0.017 mmol), MeOH (3 mL) and DCM (1 mL), sealed and irradiated in a microwave reactor at 90° C. for 30 min. The mixture was concentrated and purified by SiO₂ chromatography eluting with 5% MeOH/DCM to afford 40 mg (57%) 84.

step 2—A reaction vessel was charged with 84 (40 mg, 0.077 mmol), 48% HBr (0.1 mL) and HOAc (2 mL), capped and heated overnight at 65° C. The mixture was neutralized with sat'd. aq. NaHCO₃ and extracted with EtOAc. The organic solution was washed with brine, dried (MgSO₄), filtered and concentrated. The crude material was purified by HPLC to afford 3.2 mg of I-6.

### Example 12

### 3-[3-tert-Butyl-2-methoxy-5-(2-oxo-1,2-dihydro-pyridin-3-yl)-phenyl]-2H-isoquinolin-1-one (I-12)

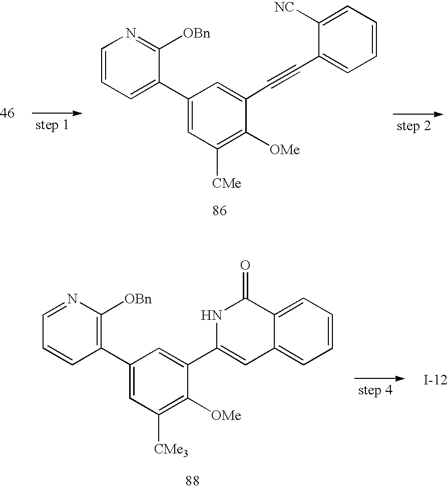

step 1—A solution of 46 (300 mg, 0.81 mmol), 2-iodobenzonitrile (222 mg, 0.97 mmol), CuI (8 mg, 0.04 mmol), PdCl₂(PPh₃)₂ (57 mg, 0.081 mmol), TEA (3 mL) in DMF (6 mL) was stirred at 75° C. for 3 h. The mixture was cooled, quenched with aqueous 1N HCl and twice extracted with EtOAc. The organic solution was washed sequentially with H₂O and brine, dried (MgSO₄), filtered and concentrated. The crude material was purified by SiO₂ chromatography eluting with an EtOAc/hexane gradient (10% to 50% EtOAc) to afford 235 mg (61.5%) of 86.

step 2—A solution of 86 (263 mg, 0.56 mmol) and 81 (30 mg, 0.07 mmol) in EtOH (20 mL) was heated at 85° C. overnight. The reaction mixture was cooled and concentrated. The crude material was purified by SiO₂ chromatography eluting with an EtOAc/hexane gradient (5% to 60% EtOAc) to afford 138 mg (50%) of 88.

step 3—A reaction vessel was charged with 88 (50 mg, 0.1 mmol), 48% HBr (0.1 mL) and HOAc (2 mL), capped and stirred at RT overnight. The mixture was neutralized with sat'd. aq. NaHCO₃ and extracted EtOAc. The combined extracts were washed with brine, dried (MgSO₄), filtered and concentrated. The crude product was purified by SiO₂ chromatography eluting with 6% MeOH/DCM to afford 27 mg (68%) of I-12.

### Example 13

### N-{2-[3-tert-Butyl-2-methoxy-5-(2-oxo-1,2-dihydro-pyridin-3-yl)-phenyl]-4-oxo-3,4-dihydro-quinazolin-6-yl}-methanesulfonamide (I-13)

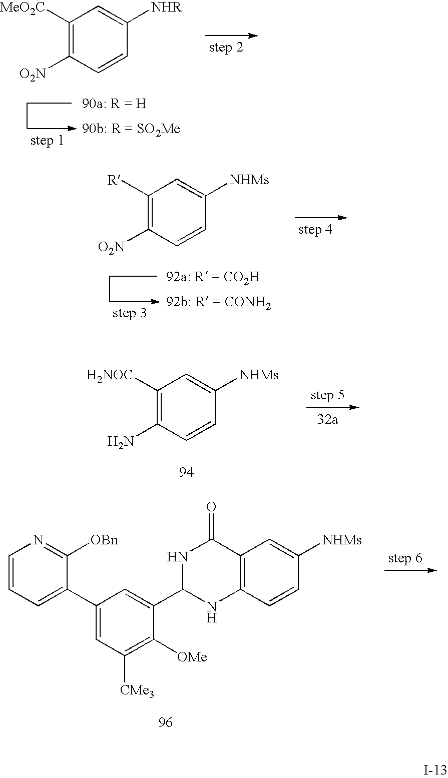

step 1—To a solution of 90a (992 mg, 5.06 mmol), pyridine (2.0 mL, 25.0 mmol) in DCM (25 mL) cooled to 5° C. was added dropwise over a period of 20 min a solution of methanesulfonyl chloride (430 μL, 630 mg, 5.60 mmol) in DCM (5 mL). The reaction mixture was stirred at RT overnight and was poured into aq. 1N HCl. The solution was extracted with EtOAc, washed with brine, dried (MgSO₄), filtered and concentrated to afford 1.15 g (82%) of 90b which was used without further purification.

step 2—A solution of 90b from step 1 was dissolved in a mixture of 1M LiOH (4 mL), THF (10 mL), MeOH (10 mL) and H₂O (6 mL) and heated to 65° C. overnight. The solvent was evaporated and the residue was dissolved in water and washed with Et₂O. The aqueous solution was made acidic with 1N HCl and extracted with EtOAc. The organic extracts were washed with brine, dried (MgSO₄), filtered and concentrated to afford 0.888 g (81%) of 92a.

step 3—To a suspension of 92a (888 mg, 3.4 mmol) in DCE was added a drop of DMF, followed by thionyl chloride (1.48 mL, 20 mmol). The suspension was stirred at 65° C. for 6 h upon which the suspension turned into a clear light yellow solution. The solution was allowed to stir overnight at RT. Excess thionyl chloride and solvent were evaporated. The residual solid was then added to concentrated NH₄OH. After ten minutes the ammonium solution was evaporated. The residue was partitioned between H₂O and EtOAc. The solid was filtered, washed with water and dried to afford 138 mg of 92b. The filtrate was basified to pH 5 and the EtOAc solution was separated. The organic solution was washed with brine, dried (MgSO₄), filtered and concentrated to afford 650 mg (74% yield) of 92b.

step 4—To a mixture of 92b (332 mg, 1.28 mmol) and NH₄Cl (680 mg, 128 mmol) in MeOH (50 mL) and H₂O (25 mL) at 70° C. was added Fe powder (360 mg, 6.4 mmol) over 60 min. After the addition was complete, the mixture was stirred at 70° C. for 2 h then cooled. The mixture was filtered through a plug of CELITE and washed with MeOH. The filtrate was concentrated and partitioned between H₂O and EtOAc. The organic solution was washed with brine, dried (MgSO₄), filtered and concentrated to afford 200 mg (68%) of 94.

step 5—A mixture of 94 (200 mg, 8.7 mmol), 32a (39 mg, 1.05 mmol) and p-TsOH.H₂O (15 mg, 0.087 mmol) in MeOH (30 mL) was heated at reflux overnight, cooled and concentrated. The crude product was purified by SiO₂ chromatography eluting with 5% MeOH/DCM to afford 30 mg (6%) of 96 along with 40 mg (9.3%) of the debenzylated derivative.

step 6—A tube was charged with 96 (30 mg, 0.05 mmol), FeCl₃ (16.5 mg, 0.1 mmol) in H₂O (5 mL) and MeOH (3 mL), sealed and irradiated in a microware reactor at 110° C. for 1 h. The reaction was cooled then partitioned between H₂O and EtOAc. The organic solution was washed with brine, dried (MgSO₄), filtered and concentrated. The crude material was purified by SiO₂ chromatography eluting with 10% MeOH/DCM to afford 11 mg (44%) of I-13.

### Example 14

### N-{3-[3-tert-Butyl-5-(2,4-dioxo-1,2,3,4-tetrahydro-pyrimidin-5-yl)-2-methoxy-phenyl]-1-oxo-1H-isochromen-7-yl}-methanesulfonamide (100)

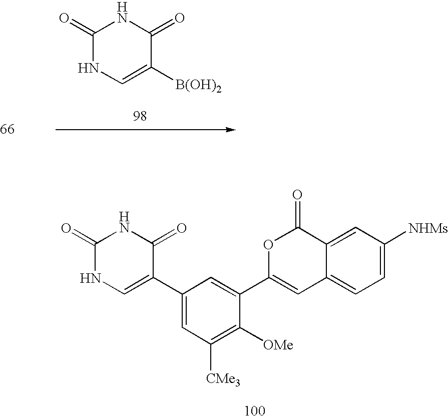

To a mixture of 66 (0.05 g, 0.114 mmol), 98 (0.036 g, 0.228 mmol, CASRN 70523-22-7), Na₂CO₃ (36 mg, 0.342 mmol) in MeOH (3 mL) and DCM (1 mL) was added Pd(PPh₃)₄ (13 mg, 0.011 mmol). The solution mixture was purged with argon for 2 min and then irradiated in a microwave synthesizer at 125° C. for 40 min. The reaction mixture is cooled to RT, diluted with DCM and filtered through CELITE. The filtrate is concentrated and the crude mixture is purified on a preparative SiO₂ TLC plate developed with 6% MeOH/DCM plate to afford 100.

### Example 15

### N-{3-[3-tert-Butyl-2-methoxy-5-(3-oxo-2,3-dihydro-pyridazin-4-yl)-phenyl]-1-oxo-1H-isochromen-7-yl}-methanesulfonamide (104)

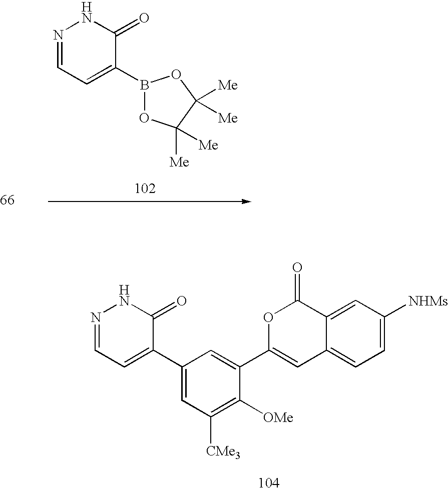

A microwave vial is charged with 66 (0.28 mmol), 102 (0.31 mmol), Pd(PPh₃)₄ (0.028 mmol), Na₂CO₃ (1 mmol), MeOH (3 mL) and DCM (1 mL), flushed with Ar and sealed. The vial is irradiated in a microwave synthesizer for at 115° C. for 30 min. The reaction mixture is cooled, concentrated and the residue partitioned between DCM (50 mL) and aq. acetate buffer at pH 4.6. The aqueous layer is extracted with DCM and the combined extracts are dried (Na₂SO₄), filtered and evaporated. The crude product is purified by SiO₂ chromatography to afford 104.

### 4-(4,4,5,5-Tetramethyl-[1,3,2]dioxaborolan-2-yl)-2H-pyridazin-3-one (102)—

step a—A 1 L round-bottom flask was charged with 4-chloro-5-hydrazinyl-3(2H)-pyridazinone (8.0 g, 50 mmol), CuSO₄.5H₂O (26.12 g, 10.5 mmol) and H₂O (300 mL) and the mixture was stirred and heated at reflux overnight. The reaction was cooled to 0° C. and an aq. solution of NaOH was added until the pH was 4. The aqueous layer was thrice extracted with EtOAc (500 mL each). The combined extracts were dried (Na₂SO₄), filtered and evaporated. The remaining aqueous phase was adjusted to pH of 2 with 37% HCl and the solution extracted six times with EtOAc. The extracts were combined, dried (Na₂SO₄), filtered and evaporated to afford 4.75 g of 4-chloro-2H-pyridazin-3-one (106)

step b—A microwave vial was charged with 106 (0.400 g, 3 mmol), bis-(pinacolato)diboron (0.934 g, 4 mmol), dicyclohexyl[2′,4′,6′-tris(1-methylethyl)[1,1′-biphenyl]-2-yl]-phosphine (X-Phos, 0.058 g, 0.12 mmol), Pd₂(dba)₃ (0.056 g, 0.061 mmol) and KOAc (0.902 g, 9 mmol) and the flask was evacuated and back-filled with Ar and sealed. Dioxane (6 mL) was added and the reaction heated at 110° C. overnight. The reaction mixture was cooled to RT and extracted with EtOAc (120 mL). The organic extract was washed sequentially with H₂O (10 mL) and brine (10 mL), dried (Na₂SO₄), filtered and evaporated. The crude product was triturated with Et₂O to afford 0.217 g of 102.

### Example 16

### N-{3-[3-tert-Butyl-2-methoxy-5-(3-oxo-3,4-dihydro-pyrazin-2-yl)-phenyl]-1-oxo-1H-isochromen-7-yl}-methanesulfonamide (112)

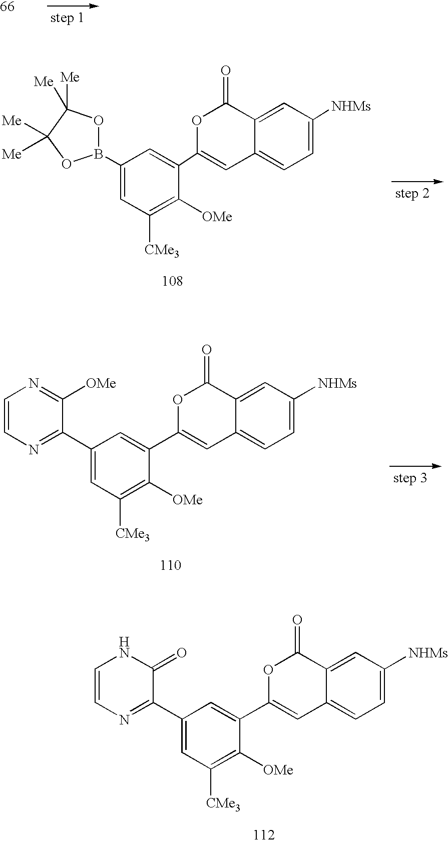

step 1—A flask is charged with 66 (0.329 mmol), bis-(pinacolato)diboron (0.36 mmol), KOAc (0.988 mmol), PdCl₂(PPh₃)₄ (0.015 g) and dioxane (6 mL) and the resulting mixture is heated at reflux for 2 h. The solution is cooled to RT and partitioned between H₂O and EtOAc. The organic extract is washed with brine, dried (Na₂SO₄), filtered and evaporated. The crude boronate ester is purified by SiO₂ chromatography eluting with EtOAc/hexane to 108.

step 2—A flask is charged with 108 (0.332 mmol), 2-chloro-3-methoxy-pyrazine (0.329 mmol), Na₂CO₃ (0.32 g, 0.997 mmol), Pd(Ph₃)₄ (0.038 g) and DCM/MeOH (3:1) and the resulting solution is heated to 110° C. for 30 min. The solution is cooled to RT, filtered and the crude product purified by SiO₂ chromatography to afford 110.

step 3—Cleavage of the methyl ether to afford 112 is carried out in accord with the procedure described in step 2 of Example 7.

### Example 17

### N-{3-[3-tert-Butyl-2-methoxy-5-(6-oxo-1,6-dihydro-pyrimidin-5-yl)-phenyl]-1-oxo-1H-isochromen-7-yl}-methanesulfonamide

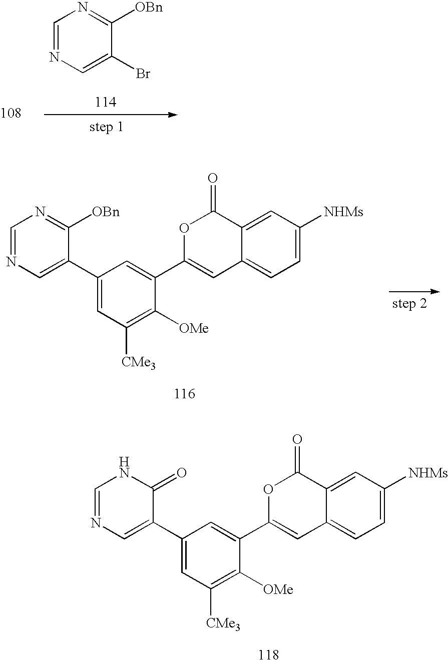

4-benzyloxy-5-bromo-pyrimidine (114)—To a suspension of 5-bromo-4(3H)-pyrimidinone (1.00 g, 5.6 mmol, CASRN 19808-30-1), 50% silver carbonate on CELITE (3.467 g, 6 mmol) and toluene (30 mL) was added benzyl bromide (0.75 mL, 6 mmol) and the resulting mixture heated at 125° C. for 1 h. The reaction was cooled and filtered through a glass microfiber filter which was rinsed with toluene. The filtrate was evaporated and the residue purified by SiO₂ chromatography eluting with an EtOAc/hexane gradient (0 to 10% EtOAc) to afford 0.140 g of 132,

step 1—Suzuki coupling of 116 and 114 is carried out in accord with the procedure described in step 5 of example 1. The crude product is purified by SiO₂ chromatography.

step 2—Cleavage of the methyl ether to afford 118 is carried out in accord with the procedure described in step 2 of Example 7.

### Example 18

### N-{3-[2-Methoxy-3-(1-methyl-cyclopropyl)-5-(2-oxo-1,2-dihydro-pyridin-3-yl)-phenyl]-1-oxo-1H-isochromen-7-yl}-methanesulfonamide

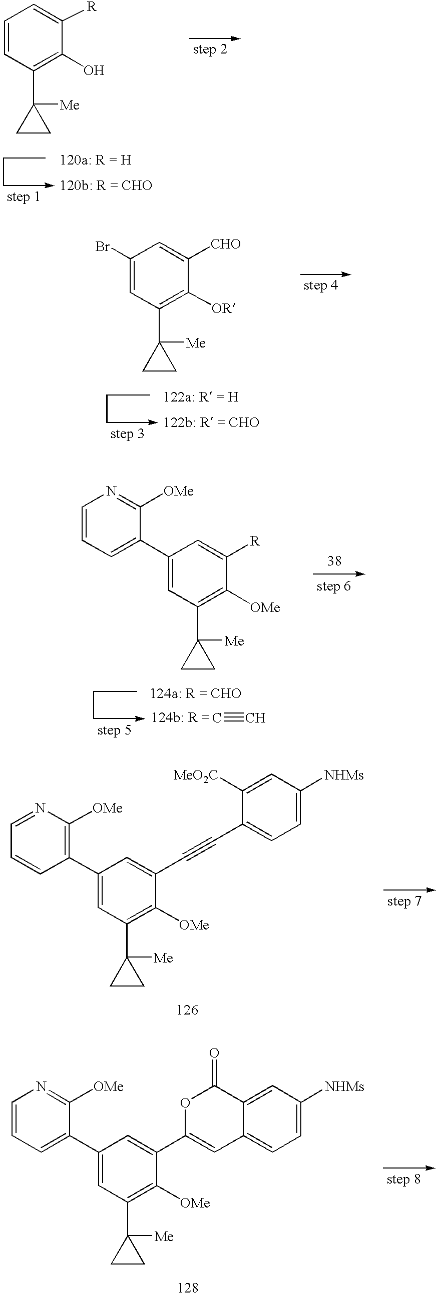

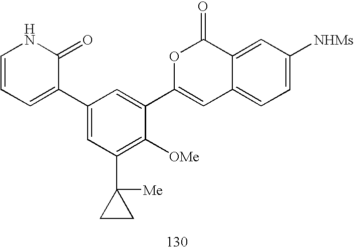

step 1—To a solution of 2-(1-methylcyclopropyl)phenol (120a, 0.55 g, 3.4 mmol; CASRN 433684-77-6) in MeCN (7 mL) was added paraformaldehyde (0.68 g, 23 mmol), MgCl₂ (0.48 g, 0.051 mmol) and TEA (1.3 g, 13 mmol). The mixture was stirred and heated at reflux for 5 h. After cooling to RT, the reaction mixture was partitioned between DCM and 1M aqueous HCl, and the organic extracts were dried (Na₂SO₄), filtered and concentrated. The crude residue was purified by SiO₂ chromatography eluting with EtOAc/hexane to afford 0.34 g (58%) of 2-hydroxy-3-(1-methylcyclopropyl)-benzaldehyde (120b) as a light yellow oil.

step 2: To a solution 120b (0.34 g, 1.9 mmol) in DCM-MeOH (3:2, 20 mL) was added tetrabutylammonium tribromide (0.98 g, 2.0 mmol) and the resulting mixture was stirred at RT for 75 min. The solvent was removed under reduced pressure and the residue was partitioned between EtOAc and water. The EtOAc layer was washed sequentially with water and brine, dried (Na₂SO₄), filtered and concentrated. The crude residue was purified by SiO₂ chromatography eluting with EtOAc/hexane to afford 0.45 g (91%) of 5-bromo-2-hydroxy-3-(1-methylcyclopropyl)benzaldehyde (122a) as a light yellow solid.

step 3—To a solution of 122a (0.44 g, 1.7 mmol) in DMF (4 mL) was added K₂CO₃ (0.60 g, 4.4 mmol) and iodomethane (0.32 g, 2.3 mmol). The resulting mixture was stirred at 60° C. for 2 h. The reaction mixture was cooled to RT and partitioned between water and Et₂O. The organic layer was washed sequentially with water and brine, dried (Na₂SO₄), filtered and concentrated to afford 0.47 g (96%) of 5-bromo-2-methoxy-3-(1-methylcyclopropyl)benzaldehyde(122b) as a light yellow solid.

step 4—Suzuki coupling of 122b and 2-methoxy-pyridin-3-yl boronic acid (CASRN 163105-90-6) to afford 124a is carried out in accord with the procedure described in step 5 of example 1. The crude product is purified by SiO₂ chromatography.

step 5—Conversion of 124a to the acetylene 124b is carried out in accord with the procedure described in step b of Example 4.

step 6—The palladium-catalyzed cross coupling of 124b and 38 is carried out in accord with the procedure described in step 3 of Example 4.

step 7—The AuCl₃ catalyzed cyclization of the acetylenic ester 126 to afford the isochromene 128 is carried out in accord with the procedure described in step 4 of Example 4.

step 8—Cleavage of the methyl ether to afford 130 is carried out in accord with the procedure described in step 2 of Example 7.

Compound I-15 was prepared from analogously starting from 341-difluoromethyl-cyclopropyl)-2-methoxy-5-(2-methoxy-pyridin-3-yl)-benzaldehyde which was prepared as described by K. A. Bramfeld et al. WO2010/0010017 (p. 181).

### Example 19

### N-{3-[3,3-Dimethyl-5-(2-oxo-1,2-dihydro-pyridin-3-yl)-2,3-dihydro-benzofuran-7-yl]-1-oxo-1H-isochromen-7-yl}-methanesulfonamide (146)

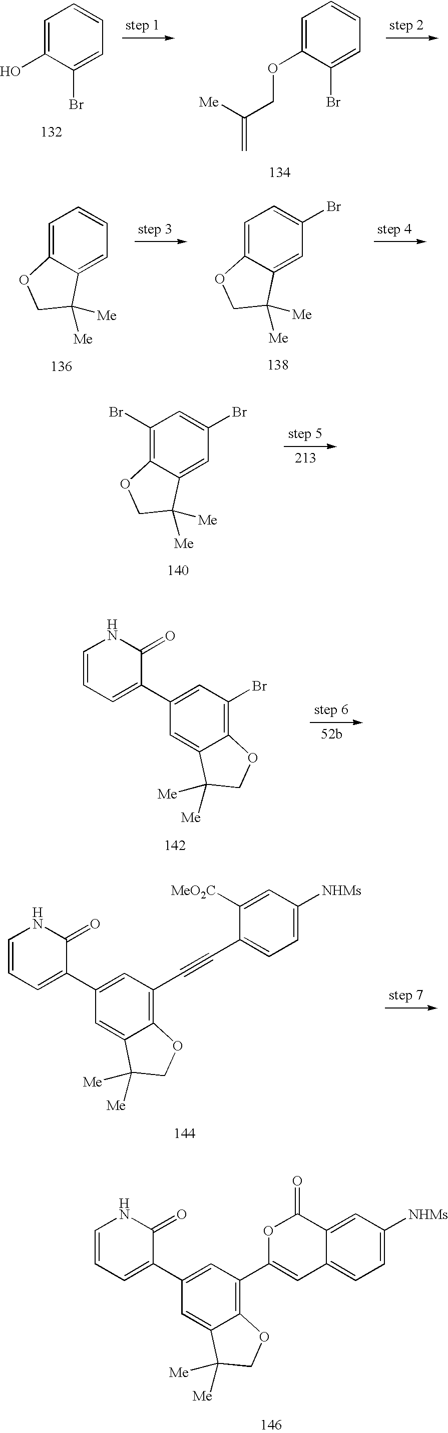

step 1—To a solution of 260 (2.457 g, 14 mmol) and acetone (75 mL) was added K₂CO₃ (4.907 g, 36 mmol) and 3-bromo-1-methylpropene (2.0 mL, 20 mmol) and the resulting solution was heated at reflux overnight. The reaction mixture was cooled and concentrated in vacuo. The residue was partitioned between EtOAc (150 mL) and H₂O (40 mL) The aqueous phase was extracted with EtOAc and the combined organic extracts were sequentially washed with H₂O and brine, dried (Na₂SO₄), filtered and concentrated in vacuo. The residue was purified by SiO₂ chromatography eluting with a EtOAc/hexane gradient (0 to 5% EtOAc) to afford 3.34 g (98.5%) of 262.

step 2—To a solution of 262 (3.33 g, 15 mmol) and benzene (150 mL) in a dried flask was added sequentially Bu₃SnH (6.625 g, 22 mmol) and AIBN (0.241 g) and the resulting solution heated at reflux overnight. The reaction mixture was cooled to RT, a 10% KF solution was added and the resulting two-phase mixture stirred vigorously for 2 h. The phases were separated and the organic phase was sequentially washed with sat'd NaHCO₃ (50 mL) and brine. The combined organic extracts were dried (Na₂SO₄), filtered and evaporated. The crude product was purified by SiO₂ chromatography eluting with a DCM/hexane gradient (0 to 10% DCM to afford 1.855 g (85%) of 264.

step 3—To a solution of 264 (0.700 g, 5 mmol) and DMF (50 mL) in a dried flask was added NBS (1.765 g, 10 mmol) and the reaction was stirred overnight at RT. The reaction mixture was partitioned between H₂O (30 mL) and Et₂O (150 mL). The aqueous layer was separated and extracted with Et₂O (150 mL). The organic extracts were thrice washed with H₂O than once with brine. The combined organic extracts were dried (Na₂SO₄), filtered and concentrated in vacuo. The residue was adsorbed on SiO₂, added to the top of a SiO₂ column and eluted with hexanes to afford 0.9260 (90%) of 266.

step 4—To a solution of 266 (0.956 g, 4 mmol) and HOAc (8.0 mL) cooled to 0° C. was added a dropwise solution of Br₂ (320 μL, 6 mmol) and HOAc (2 mL) over a 10 min period. The reaction mixture was stirred overnight at RT. The reaction was quenched by addition of 10% Na₂S₂O₃ (10 mL) then HOAc was removed in vacuo. The residue was partitioned between Et₂O (100 mL) and sat'd. aq.NaHCO₃ (20 mL). The aqueous layer was separated and extracted with Et₂O (100 mL). The organic extracts were washed twice with sat'd. NaHCO₃ (20 mL) and once with H₂O. The combined extracts were dried (Na₂SO₄), filtered and evaporated. The residue was adsorbed on SiO₂, added to the top of a SiO₂ column and eluted with hexanes to afford 1.22 (95%) of 268.

step 5—The palladium-catalyzed coupling of 268 and 213 was carried out in accord with the procedure in step 4 of example 38. The product was purified by SiO₂ chromatography eluting with a EtOAc/hexane gradient (0 to 80% EtOAc) to afford 270 wherein coupling occurred selectively at the 5-bromo substituent.

step 6—The palladium-catalyzed cross coupling of 142 and 52b to afford 144 is carried out in accord with the procedure described in step 2 of Example 6.

step 7—The AuCl₃ catalyzed cyclization of the acetylenic ester 144 to afford the isochromene 146 is carried out in accord with the procedure described in step 3 of Example 6.

N-{3-[3,3-Dimethyl-7-(2-oxo-1,2-dihydro-pyridin-3-yl)-2,3-dihydro-benzofuran-5-yl]-1-oxo-1H-isochromen-7-yl}-methanesulfonamide (148) is prepared analogously except the sequence of the coupling steps is reversed such that coupling with of 52b and 140 and the AuC13 catalyzed cyclization (step 7) will be carried out prior to coupling with 28 in accord with the procedure in step 5 to afford 148.

### Example 11

HCV NS5B RNA Polymerase Activity

The enzymatic activity of HCV polymerase (NS5B570n-Con1) was measured as the incorporation of radiolabeled nucleotide monophosphates into acid insoluble RNA products. Unincorporated radiolabeled substrate was removed by filtration and scintillant was added to the washed and dried filter plate containing radiolabeled RNA product. The amount of RNA product generated by NS5B570-Con1 at the end of the reaction was directly proportional to the amount of light emitted by the scintillant.

The N-terminal 6-histidine tagged HCV polymerase, derived from HCV Con1 strain, genotype 1b (NS5B570n-Con1) contains a 21 amino acid deletion at the C-terminus relative to the full-length HCV polymerase and was purified from E. coli strain BL21(DE) pLysS. The construct, containing the coding sequence of HCV NS5B Con1 (GenBank accession number AJ242654) was inserted into the plasmid construct pET17b, downstream of a T7 promoter expression cassette and transformed into E. coli. A single colony was grown overnight as a starter culture and later used inoculate 10 L of LB media supplemented with 100 μg/mL ampicillin at 37° C. Protein expression was induced by the addition of 0.25 mM isopropyl-β-D-thiogalactopyranoside (IPTG) when optical density at 600 nM of the culture was between 0.6 and 0.8 and cells were harvested after 16 to 18 h at 30° C. NS5B570n-Con1 was purified to homogeneity using a three-step protocol including subsequent column chromatography on Ni-NTA, SP-Sepharose HP and Superdex 75 resins.

Each 50 μl enzymatic reaction contained 20 nM RNA template derived from the complementary sequence of the Internal Ribosome Entry Site (cIRES), 20 nM NS5B570n-Con1 enzyme, 0.5 μCi of tritiated UTP (Perkin Elmer catalog no. TRK-412; specific activity: 30 to 60 Ci/mmol; stock solution concentration from 7.5×10-5 M to 20.6×10-6 M), 1 μM each ATP, CTP, and GTP, 40 mM Tris-HCl pH 8.0, 40 mM NaCl, 4 mM DTT (dithiothreitol), 4 mM MgC12, and 5 μl of compound serial diluted in DMSO. Reaction mixtures were assembled in 96-well filter plates (cat #MADVNOB, Millipore Co.) and incubated for 2 h at 30° C. Reactions were stopped by addition of 10% final (v/v) trichloroacetic acid and incubated for 40 min at 4° C. Reactions were filtered, washed with 8 reaction volumes of 10% (v/v) trichloroacetic acetic acid, 4 reaction volumes of 70% (v/v) ethanol, air dried, and 25 μl of scintillant (Microscint 20, Perkin-Elmer) was added to each reaction well.

The amount of light emitted from the scintillant was converted to counts per minute (CPM) on a Topcount® plate reader (Perkin-Elmer, Energy Range: Low, Efficiency Mode Normal, Count Time: 1 min, Background Subtract: none, Cross talk reduction: Off).

Data was analyzed in Excel® (Microsoft®) and ActivityBase® (idbs®). The reaction in the absence of enzyme was used to determine the background signal, which was subtracted from the enzymatic reactions. Positive control reactions were performed in the absence of compound, from which the background corrected activity was set as 100% polymerase activity. All data was expressed as a percentage of the positive control. The compound concentration at which the enzyme-catalyzed rate of RNA synthesis was reduced by 50% (IC₅₀) was calculated by fitting equation (i) to

$$Y = \% Min + \frac{( \% Max - \% Min )}{[ 1 + \frac{X}{{( {IC}_{50} )}^{S}} ]} \qquad ( i )$$

the data where “Y” corresponds to the relative enzyme activity (in %), “% Min” is the residual relative activity at saturating compound concentration, “% Max” is the relative maximum enzymatic activity, “X” corresponds to the compound concentration, and “S” is the Hill coefficient (or slope).

### Example 12

### HCV Replicon Assay

This assay measures the ability of the compounds of formula Ito inhibit HCV RNA replication, and therefore their potential utility for the treatment of HCV infections. The assay utilizes a reporter as a simple readout for intracellular HCV replicon RNA level. The Renilla luciferase gene was introduced into the first open reading frame of a genotype 1b replicon construct NK5.1 (N. Krieger et al., J. Virol. 2001 75(10):4614), immediately after the internal ribosome entry site (IRES) sequence, and fused with the neomycin phosphotransferase (NPTII) gene via a self-cleavage peptide 2A from foot and mouth disease virus (M. D. Ryan & J. Drew, EMBO 1994 13(4):928-933). After in vitro transcription the RNA was electroporated into human hepatoma Huh7 cells, and G418-resistant colonies were isolated and expanded. Stably selected cell line 2209-23 contains replicative HCV subgenomic RNA, and the activity of Renilla luciferase expressed by the replicon reflects its RNA level in the cells. The assay was carried out in duplicate plates, one in opaque white and one in transparent, in order to measure the anti-viral activity and cytotoxicity of a chemical compound in parallel ensuring the observed activity is not due to decreased cell proliferation or due to cell death.

HCV replicon cells (2209-23), which express Renilla luciferase reporter, were cultured in Dulbecco's MEM (Invitrogen cat no. 10569-010) with 5% fetal bovine serum (FBS, Invitrogen cat. no. 10082-147) and plated onto a 96-well plate at 5000 cells per well, and incubated overnight. Twenty-four hours later, different dilutions of chemical compounds in the growth medium were added to the cells, which were then further incubated at 37° C. for three days. At the end of the incubation time, the cells in white plates were harvested and luciferase activity was measured by using the R. luciferase Assay system (Promega cat no. E2820). All the reagents described in the following paragraph were included in the manufacturer's kit, and the manufacturer's instructions were followed for preparations of the reagents. The cells were washed once with 100 μl of phosphate buffered saline (pH 7.0) (PBS) per well and lysed with 20 μl of 1×R. luciferase Assay lysis buffer prior to incubation at room temperature for 20 min. The plate was then inserted into the Centro LB 960 microplate luminometer (Berthold Technologies), and 100 μl of R. luciferase Assay buffer was injected into each well and the signal measured using a 2-second delay, 2-second measurement program. IC₅₀, the concentration of the drug required for reducing replicon level by 50% in relation to the untreated cell control value, can be calculated from the plot of percentage reduction of the luciferase activity vs. drug concentration as described above.

WST-1 reagent from Roche Diagnostic (cat no. 1644807) was used for the cytotoxicity assay. Ten microliter of WST-1 reagent was added to each well of the transparent plates including wells that contain media alone as blanks. Cells were then incubated for 2 h at 37° C., and the OD value was measured using the MRX Revelation microtiter plate reader (Lab System) at 450 nm (reference filter at 650 nm). Again CC₅₀, the concentration of the drug required for reducing cell proliferation by 50% in relation to the untreated cell control value, can be calculated from the plot of percentage reduction of the WST-1 value vs. drug concentration as described above.

TABLE II

| Compound | HCV Replicon Activity | Cytotoxic Activity |
|---|---|---|
| Number | IC₅₀ (μM) | CC₅₀ (μM) |
| I-11 | 0.001 | 42.6 |

### Example 13

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound according to formula I wherein: 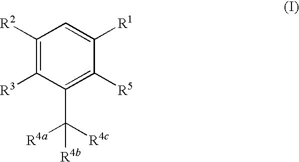
R¹ is selected from the group consisting of A-1, A-2, A-3 and A-4 wherein the dotted line is either a single or a double bond; 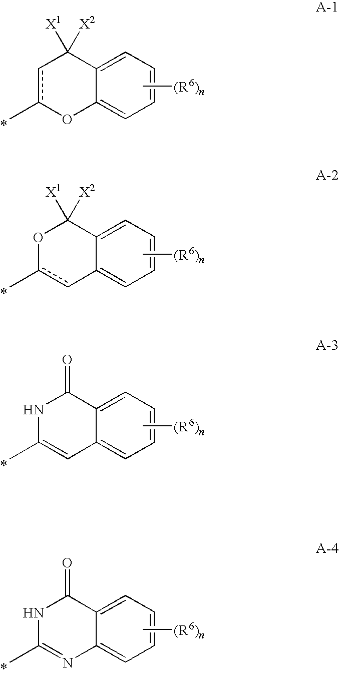
X¹ and X² each are hydrogen or X¹ and X² together are oxo;
R² is a heteroaryl radical selected from the group consisting of 2-oxo-1,2-dihydro-pyridin-3-yl, 3-oxo-3,4-dihydro-pyrazin-2-yl, 3-oxo-2,3-dihydro-pyridazin-4-yl, 2-oxo-1,2-dihydro-pyrimidin-4-one-5-yl and 6-oxo-1,6-dihydro-[1,2,4]triazin-5-yl said heteroaryl being optionally substituted by halogen, C₁₋₆ alkyl, C₁₋₃ haloalkyl, C₁₋₆ alkoxy, optionally substituted aryl-C₁₋₃ alkyl, —X—(CH₂)ₘNR^{c}R^{d} or X—(CH₂)ₘCO₂H wherein X is oxygen or a bond, m is 1 to 5 and R^{c} and R^{d} are independently hydrogen or C₁₋₃ alkyl or R^{c} and R^{d} together with the nitrogen atom to which they are attached are a cyclic amine;
R³ is hydrogen, fluorine or R³ and R^{4a} together are CH₂—O and together with atoms to which they are attached form a 2,3-dihydrobenzofuran or an indane;
R^{4a}, R^{4b} and R^{4c} (i) when taken independently are selected independently from C₁₋₃ alkyl, C₁₋₂ alkoxy, C₁₋₂ fluoroalkyl, C₁₋₃ hydroxyalkyl, cyano or hydroxy or (ii) when taken together, R^{4a} and R^{4b} together are C₂₋₄ alkylene and R^{4c} is hydrogen, C₁₋₃ alkyl, C₁₋₂ alkoxy, halogen, C₁₋₃ hydroxyalkyl, cyano or C₁₋₂ fluoroalkyl or R^{4a} and R^{4b} together with the carbon to which they are attached are 3-oxetanyl, or tetrahydrofuran-2-yl or (iii) either R⁵ or R³ and R^{4a} together are CH₂—O or (CH₂)₂ and together with atoms to which they are attached form a 2,3-dihydro-benzofuran or an indane and R^{4b} and R^{4c} are C₁₋₃ alkyl or (iv) R^{4a}, R^{4b} and R^{4c}; along with the carbon to which they are attached are a cyclopropyl, trifluoromethyl or 2,2,2-trifluoroethyl group;
R⁵ is hydrogen, C₁₋₆ alkyl, C₁₋₆ haloalkyl, C₁₋₆ alkoxy, C₁₋₆ halo alkoxy, C₁₋₃ alkoxy-C₁₋₆ alkoxy, halogen or R⁵ and R^{4a} together are CH₂—O and together with atoms to which they are attached form a 2,3-dihydrobenzofuran or an indane;
R⁶ is halogen, C₁₋₃ acylamino-C₁₋₆ alkyl, (CH₂)ₙNR^{a}R^{b} or (CH₂)ₙCONR^{a}R^{b};
R^{a} and R^{b} are independently in each occurrence hydrogen, C₁₋₆ alkyl, C₁₋₃ haloalkyl, C₁₋₆ acyl, C₁₋₆ alkylsulfonyl, C₁₋₆ haloalkylsulfonyl, C₃₋₇ cycloalkylsulfonyl, C₃₋₇ cycloalkyl-C₁₋₃ alkyl-sulfonyl, C₁₋₆ alkoxy-C₁₋₆ alkylsulfonyl or (CH₂)₁₋₃NR^{e}R^{f} wherein R^{e} and R^{f} are independently hydrogen or C₁₋₆ alkyl or R^{e} and R^{f} together with the nitrogen to which they are attached are an optionally substituted cyclic amine;
n is independently in each occurrence zero to two; or,
a pharmaceutically acceptable salt thereof.

2. The compound according to claim 1 wherein R¹ is A-1, X¹ and X² together are oxo, the dotted line represents a double bond, R³ is hydrogen and R⁵ is hydrogen or C₁₋₆ alkoxy.

3. The compound according to claim 2 wherein either (i) R^{4a}, R^{4b} and R^{4c} are methyl, or (ii) R^{4a} and R⁴⁶ together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl.

4. The compound according to claim 3 wherein R⁶ is methanesulfonylamino substituted at the 6-position.

5. The compound according to claim 1 wherein R¹ is A-1, R³ is hydrogen, X¹ and X² are hydrogen and R⁵ is hydrogen or C₁₋₆ alkoxy.

6. The compound according to claim 1 wherein R¹ is A-2, X¹ and X² together are oxo, the dotted line represents a double bond, R³ is hydrogen, and R⁵ is hydrogen or C₁₋₆ alkoxy.

7. The compound according to claim 6 wherein either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl.

8. The compound according to claim 7 wherein R⁶ is methanesulfonylamino substituted at the 7-position.

9. The compound according to claim 1 wherein R¹ is A-2, X¹ and X² are hydrogen, R³ is hydrogen and R⁵ is hydrogen or C₁₋₆ alkoxy.

10. The compound according to claim 1 wherein R¹ is A-3, the dotted line represents a double bond, R³ is hydrogen and R⁵ is hydrogen or C₁₋₆ alkoxy.

11. The compound according to claim 10 wherein either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or an indane and R^{4b} and R^{4c} are methyl.

12. The compound according to claim 11 wherein R⁶ is methanesulfonylamino substituted at the 7-position.

13. The compound according to claim 1 wherein R¹ is A-4, R³ is hydrogen and R⁵ is hydrogen or C₁₋₆ alkoxy.

14. The compound according to claim 13 wherein and either (i) R^{4a}, R^{4b} and R^{4c} are methyl, or (ii) R^{4a} and R^{4b} together are C₂ alkylene and R^{4c} is methyl, or (iii) either R⁵ or R³ and R^{4a} together are CH₂—O or (CH₂)₂ and together with atoms to which they are attached form a 2,3-dihydro-benzofuran or indane and R^{4b} and R^{4c} are methyl

15. The compound according to claim 14 wherein R⁶ is methanesulfonylamino substituted at the 7-position.

16. The compound according to claim 1 selected from the group consisting of:
N-{2-[3-tert-butyl-2-methoxy-5-(2-oxo-1,2-dihydro-pyridin-3-yl)-phenyl]-4-oxo-4H-chromen-6-yl}-methanesulfonamide,
N-{2-[3-tert-butyl-2-methoxy-5-(2-oxo-1,2-dihydro-pyridin-3-yl)-phenyl]-4H-chromen-6-yl}-methanesulfonamide,
N-{2-[3-tert-butyl-2-methoxy-5-(2-oxo-1,2-dihydro-pyridin-3-yl)-phenyl]-chroman-6-yl}-methanesulfonamide,
N-{3-[3-tert-butyl-2-methoxy-5-(2-oxo-1,2-dihydro-pyridin-3-yl)-phenyl]-1-oxo-1H-isochromen-7-yl}-methanesulfonamide,
N-{3-[3-tert-butyl-5-(2-oxo-1,2-dihydro-pyridin-3-yl)-phenyl]-1-oxo-1H-isochromen-7-yl}-methanesulfonamide,
N-{3-[3-tert-butyl-5-(5-fluoro-2-oxo-1,2-dihydro-pyridin-3-yl)-2-methoxy-phenyl]-1-oxo-1H-isochromen-7-yl}-methanesulfonamide,
3-[3-tert-butyl-4-methoxy-5-(1-oxo-1H-isochromen-3-yl)-phenyl]-1H-pyridin-2-one
N-{3-[3-tert-butyl-2-methoxy-5-(6-methoxy-2-oxo-1,2-dihydro-pyridin-3-yl)-phenyl]-1-oxo-1H-isochromen-7-yl}-methanesulfonamide,
N-{3-[3-tert-butyl-2-methoxy-5-(2-oxo-1,2-dihydro-pyridin-3-yl)-phenyl]-1-oxo-isochroman-7-yl}-methanesulfonamide,
N-{3-[3-tert-butyl-2-methoxy-5-(2-oxo-1,2-dihydro-pyridin-3-yl)-phenyl]-isochroman-7-yl}-methanesulfonamide,
N-{3-[3-tert-butyl-2-methoxy-5-(2-oxo-1,2-dihydro-pyridin-3-yl)-phenyl]-1-oxo-1,2-dihydro-isoquinolin-7-yl}-methanesulfonamide,
N-{3-[3-(1-difluoromethyl-cyclopropyl)-5-(5-fluoro-2-oxo-1,2-dihydro-pyridin-3-yl)-2-methoxy-phenyl]-1-oxo-1H-isochromen-7-yl}-methanesulfonamide,
3-[3-tert-butyl-2-methoxy-5-(2-oxo-1,2-dihydro-pyridin-3-yl)-phenyl]-2H-isoquinolin-1-one, and,
N-{2-[3-tert-butyl-2-methoxy-5-(2-oxo-1,2-dihydro-pyridin-3-yl)-phenyl]-4-oxo-3,4-dihydro-quinazolin-6-yl}-methanesulfonamide;
or, a pharmaceutically acceptable salt thereof.

17. A method for treating a Hepatitis C Virus (HCV) infection comprising administering to a patient in need thereof, a therapeutically effective quantity of a compound according to claim 1.

18. The method of claim 17 further co-comprising administering at least one immune system modulator and/or at least one antiviral agent that inhibits replication of HCV.

19. The method of claim 18 wherein the immune system modulator is an interferon, interleukin, tumor necrosis factor or colony stimulating factor.

20. The method of claim 19 wherein the immune system modulator is an interferon or chemically derivatized interferon.

21. The method of claim 18 wherein the antiviral compound is selected from the group consisting of a HCV protease inhibitor, another HCV polymerase inhibitor, a HCV helicase inhibitor, a HCV primase inhibitor and a HCV fusion inhibitor.

22. A method for inhibiting replication of HCV in a cell be delivering a compound according to claim 1.

23. A composition comprising a compound according to claim 1 admixed with at least one pharmaceutically acceptable carrier, diluent or excipient.
